(19) United States
(12) Patent Application Publication
(10) Pub. No.: US 2025/0387465 A1
(43) Pub. Date: Dec. 25, 2025

(54) RSV VACCINE AS WELL AS PREPARATION METHOD THEREOF AND USE THEREOF

(71) Applicants: Beijing Genevax Biotechnology Co., Ltd., Beijing (CN); Genevax (Langfang) Biotechnology Co., Ltd., Langfang (CN); Genevax (Hebei) Biotechnology Co., Ltd, Langfang (CN)

(72) Inventors: Xiliang Wang, Beijing (CN); Jincan Zhang, Beijing (CN); Jinxia Cheng, Beijing (CN); Li Wang, Beijing (CN); Shichong Li, Beijing (CN); Libo Wang, Beijing (CN)

(21) Appl. No.: 19/245,442

(22) Filed: Jun. 23, 2025

Related U.S. Application Data
(63) Continuation of application No. PCT/CN2023/140371, filed on Dec. 20, 2023.

(30) Foreign Application Priority Data
Dec. 22, 2022 (CN) 202211654193.3
Aug. 25, 2023 (CN) 202311083641.3
Dec. 8, 2023 (CN) 202311686080.6

(51) Int. Cl. A61K 39/155; A61K 9/51; A61K 39/00; A61P 37/04; C07K 14/005

(52) U.S. Cl. CPC A61K 39/155; A61K 9/5123; A61P 37/04; C07K 14/005; A61K 2039/53; A61K 2039/55505; A61K 2039/55561; C12N 2760/18522; C12N 2760/18534

(57) ABSTRACT

Provided are an RSV vaccine as well as a preparation method therefor and the use thereof. A Pre-F related sequence of an RSV and ferritin nanoparticles are subjected to mutation design, and a Pre-F mutant protein and a ferritin mutant are fused and expressed in eukaryotic cells, so as to obtain ferritin-PreF fusion protein nanoparticles having multiple Pre-F displayed on the surfaces in a centralized manner, and the amino acid sequence of the ferritin-PreF fusion protein nanoparticles is any one of SEQ ID No. 20-27.

### CROSS-REFERENCE TO RELATED APPLICATIONS

The present application is a continuation application of PCT application No. PCT/CN2023/140371, filed Dec. 20, 2023, which claims priority to Chinese Patent Application No. 202211654193.3, filed Dec. 22, 2022, Chinese Patent Application No. 202311083641.3, filed Aug. 25, 2023, and Chinese Patent Application No. 202311686080.6, filed Dec. 8, 2023, the entire contents of which are incorporated herein by reference and form part of the present application.

### REFERENCE TO SEQUENCE LISTING

This application includes a Sequence Listing filed electronically as an XML file named “Sequence listing_BELLDIM-25001-USPT.xml”, created on Jun. 20, 2025, with a size of 63,589 bytes. The Sequence Listing is incorporated herein by reference.

### TECHNICAL FIELD

The present disclosure relates to the biotechnology field, particularly to a RSV vaccine as well as its preparation and application.

### TECHNICAL BACKGROUND

Respiratory Syncytial Virus (RSV) was first discovered in 1955. It belongs to the family Paramyxoviridae, subfamily Pneumovirinae, genus Pneumovirus, and can be classified into two subtypes, A and B, based on the sequence of the G protein. RSV is a non-segmental, negative strand RNA virus with a genome length of 15.2 kb, containing 10 genes and encoding 11 proteins, including non-structural proteins (NS1, NS2), nucleoprotein (N), phosphoprotein (P), matrix protein (M), RNA-dependent RNA polymerase (L), transcription elongation factor (M2-1), regulatory factor (M2-2), and three envelope glycoproteins (attachment protein (G), fusion protein (F), and small hydrophobic protein (SH)).

RSV is a viral pathogen causing respiratory tract infections (RTIs), primarily causing lower respiratory tract infections. A high percentage of patients develop severe symptoms (such as bronchiolitis and pneumonia), often require hospitalization and are associated with high mortality rates. RSV spreads through person-to-person contact, inhalation of droplets from coughing or sneezing, or contact with contaminated surfaces. It mainly infects epithelial cells in the nasal cavity and large and small airways of the lungs, as well as alveolar macrophages and other lung cell types, and can induce cell fusion to form syncytia.

Vaccine development is currently the most concentrated area for RSV prevention and treatment. As early as the 1960s, a formalin-inactivated RSV vaccine was developed and entered clinical trials. It was the first RSV vaccine to enter clinical trials. However, this vaccine not only failed to provide protection against RSV but also caused enhanced respiratory disease (ERD) in vaccinated children upon subsequent natural infection, leading to increased hospitalization rates and even fatalities. Consequently, the vaccine was never approved for clinical use.

In recent years, with advancements in reverse genetics, vaccinology, molecular virology, genomics, and immunology, significant breakthroughs have been made in RSV vaccine research. Various types of RSV vaccines, such as live-attenuated vaccines, inactivated vaccines, chimeric vector vaccines, subunit vaccines, virus-like particle vaccines, replication-deficient viral vector vaccines, and nucleic acid vaccines, have shown clinical potential. However, to date, no effective RSV vaccine has been approved globally, primarily due to low immunogenicity and production instability.

### SUMMARY OF THE INVENTION

The objective of the present invention is to provide an RSV vaccine with high immune titer, high stability, and high safety.

To achieve this objective, the present invention first provides a protein.

The protein provided by this invention comprises mutations at one or more of the following positions in the Pre-F protein amino acid sequence: 67, 88, 110, 144, 159, 173, 202, 227, 236, 248, 289, 309, 334, 344, 370, 389, 419, and/or 468.

Preferably, the protein provided by this invention is obtained by performing at least one of the following mutations a1)-a18) on the Pre-F protein amino acid sequence:
a1) Isoleucine at position 67 is substituted with asparagine (167N);
a2) Serine at position 88 is substituted with asparagine (S88N);
a3) Cysteine at position 110 is substituted with alanine (C110A);
a4) Asparagine at position 144 is substituted with glycine, with insertion of cysteine between positions 144 and 145 (N144C);
a5) Tyrosine at position 159 is substituted with cysteine (Y159C);
a6) Deletion of cysteine at position 173 (AC173);
a7) Alanine at position 202 is substituted with cysteine (A202C);
a8) Isoleucine at position 227 is substituted with asparagine (1227N);
a9) Serine at position 236 is substituted with arginine (S236R);
a10) Serine at position 248 is substituted with cysteine (S248C);
a11) Glutamic acid at position 289 is substituted with asparagine (E289N);
a12) Serine at position 309 is substituted with asparagine (S309N);
a13) Arginine at position 334 is substituted with tyrosine (R334Y);
a14) Asparagine at position 344 is substituted with glutamic acid (N344E);
a15) Serine at position 370 is substituted with glycine (S370G);
a16) Asparagine at position 389 is substituted with cysteine (N389C);
a17) Cysteine at position 419 is substituted with tyrosine (C419Y);
a18) Arginine at position 468 is substituted with asparagine (R468N).

More preferably, the mutations in the pre-F protein include:
1. Mutation at position 88;
2. Mutations at positions 88 and 289;
3. Mutations at positions 88 and 389;
4. Mutations at positions 88, 289, 309, and 468;
5. Mutations at positions 67, 88, 144, and 389; or
6. Mutations at positions 67, 88, 110, 144, 289, 309, 389, and 468.

Further preferably, the mutations include:
1) S88N;
2) S88N and E289N;
3) S88N and N389C;
4) S88N, E289N, S309N, and R468N;
5) I67N, S88N, N144C, and N389C;
6) I67N, S88N, C110A, N144C, E289N, S309N, N389C, and R468N;
7) 167N, S88N, insertion of glycine between positions 143 and 144, N144C, and N389C.

The Pre-F protein is any one of the following:
(A1) The protein shown in SEQ ID No. 1;
(A2) A fusion protein obtained by linking a tag to the N-terminus and/or C-terminus of the protein in (A1);
(A3) A protein derived from any one of (A1)-(A2) by substitution, deletion, and/or addition of one or several amino acid residues while retaining the same function;
(A4) A protein with at least 98% identity to any one of (A1)-(A2) and having the same function.

Further, the protein (also referred to as the mutant Pre-F protein) is any one of the following:
(M1) The protein shown in SEQ ID No. 2, 3, 4, 5, 6, 7, 8, or 9;
(M2) A fusion protein obtained by linking a tag to the N-terminus and/or C-terminus of the protein in (M1);
(M3) A protein derived from any one of (M1)-(M2) by substitution, deletion, and/or addition of one or several amino acid residues while retaining the same function;
(M4) A protein with at least 98% identity to any one of (M1)-(M2) and having the same function.

To achieve the above objective, the invention further provides a fusion protein.

The fusion protein provided by the invention comprises the aforementioned protein and a ferritin mutant.

The ferritin mutant is obtained by performing at least one of the following mutations b1)-b3) on the ferritin amino acid sequence:
b1) Asparagine at position 15 is substituted with glutamine (D15Q);
b2) Serine at position 96 is substituted with asparagine (S96D);
b3) Tyrosine at position 119 is substituted with arginine (Y119R).

The ferritin is any one of the following:
(B1) The protein shown in SEQ ID No. 10;
(B2) A fusion protein obtained by linking a tag to the N-terminus and/or C-terminus of the protein in (B1);
(B3) A protein derived from any one of (B1)-(B2) by substitution, deletion, and/or addition of one or several amino acid residues while retaining the same function;
(B4) A protein with at least 98% identity to any one of (B1)-(B2) and having the same function.

Further, the ferritin mutant is any one of the following:
(N1) The protein shown in SEQ ID No. 11;
(N2) A fusion protein obtained by linking a tag to the N-terminus and/or C-terminus of the protein in (N1);
(N3) A protein derived from any one of (N1)-(N2) by substitution, deletion, and/or addition of one or several amino acid residues while retaining the same function;
(N4) A protein with at least 98% identity to any one of (N1)-(N2) and having the same function.

Further, the fusion protein is any one of the following:
(C1) The protein shown in SEQ ID No. 12, 13, 14, 15, 16, 17, 18, or 19;
(C2) A fusion protein obtained by linking a tag to the N-terminus and/or C-terminus of the protein in (C1);
(C3) A protein derived from any one of (C1)-(C2) by substitution, deletion, and/or addition of one or several amino acid residues while retaining the same function;
(C4) A protein with at least 98% identity to any one of (C1)-(C2) and having the same function.

In the fusion proteins described in (A2), (M2), (B2), (N2), or (C2), the tag refers to a polypeptide or protein fused and expressed with the target protein using DNA recombination technology to facilitate expression, detection, tracing, and/or purification of the target protein. The tag may include Flag tag, His tag, MBP tag, HA tag, myc tag, GST tag, and/or SUMO tag, and the like.

In the fusion proteins described in (A3), (M3), (B3), (N3), or (C3), the “substitution, deletion, and/or addition of one or several amino acid residues” refers to substituting, deleting, and/or adding amino acid residues outside the mutation sites specified in a1)-a18) or b1)-b3), involving no more than 10 amino acid residues.

The aforementioned proteins or fusion proteins may be artificially synthesized or obtained by synthesizing their encoding genes followed by biological expression.

The invention also provides biological materials. The biological materials provided by this invention include at least one of the following D1)-D5):
D1) A nucleic acid molecule encoding the aforementioned protein or fusion protein;
D2) An expression cassette containing the nucleic acid molecule in D1);
D3) A recombinant vector containing the nucleic acid molecule in D1) or the expression cassette in D2);
D4) A recombinant microorganism containing the nucleic acid molecule in D1), the expression cassette in D2), or the recombinant vector in B3);
D5) A recombinant cell line containing the nucleic acid molecule in D1), the expression cassette in D2), or the recombinant vector in D3).

In the aforementioned biological materials, the nucleic acid molecule encoding the protein is E1) or E2):
E1) A DNA molecule comprising nucleotides 1-1422 of SEQ ID No. 20, 21, 22, 23, 24, 25, 26, or 27; or
E2) A DNA molecule with at least 75% identity to any one of the molecules in E1) and encoding the fusion protein.

The nucleic acid molecule encoding the fusion protein is either:
F1) A DNA molecule shown in SEQ ID No. 20, 21, 22, 23, 24, 25, 26, or 27; or
F2) A DNA molecule with at least 75% identity to any one of the molecules F1) and encoding the fusion protein.

The nucleic acid molecule may be DNA (e.g., recombinant DNA) or RNA (e.g., mRNA).

Preferably, the mRNA includes:
G1) An mRNA sequence obtained by replacing T with U in nucleotides 1-1422 or the full-length sequence of any of SEQ ID No. 20-27;
G2) A degenerate or complementary sequence of G1);
G3) An mRNA molecule with at least 75% identity to any one in G1 or G2 and encoding a fusion protein with the same function.

Preferably, the mRNA includes, in addition to the coding region, a 5′ cap structure, 5′untranslated region (UTR), 3′ UTR, and/or poly (A) tail.

Preferably, the mRNA sequence is natural or modified RNA, where the modified RNA includes RNA in which natural uridine is partially or fully replaced by modified uridine.

For example, the modified RNA may be RNA in which natural uridine is fully replaced by 1-methyl-pseudouridine.

In another aspect, the invention provides an mRNA comprising a first open reading frame. The first ORF contains a nucleic acid encoding a monomeric subunit protein and at least one immunogenic part from RSV. The monomeric subunit protein is selected from the group consisting of monomeric ferritin subunit, monomeric encapsulin protein, monomeric 03-33 protein, monomeric sulfur oxygenase reductase protein, monomeric dihydropteridine synthase protein, and/or monomeric pyruvate dehydrogenase complex dihydrolipoamide acetyltransferase protein. The monomeric subunit protein expressed by the first ORF self-assembles into nanoparticles, displaying the said at least one immunogenic part on the surface of the nanoparticles.

In another aspect, the invention provides an mRNA comprising at least two ORFs. The first ORF includes a nucleic acid encoding a monomeric subunit protein selected from the group consisting of monomeric ferritin subunit, monomeric encapsulin protein, monomeric 03-33 protein, monomeric sulfur oxygenase reductase protein, monomeric dihydropteridine synthase protein, and/or monomeric pyruvate dehydrogenase complex dihydrolipoamide acetyltransferase protein. The second ORF includes a nucleic acid encoding at least one immunogenic portion from RSV. The monomeric subunit protein expressed by the first ORF self-assembles into nanoparticles, and the at least one immunogenic portion expressed by the second ORF binds to the nanoparticles expressed by the first ORF.

Preferably, the at least one immunogenic part is selected from the mutant Pre-F protein of RSV, as defined above.

Further preferably, the protein encoded by the mRNA is a fusion protein composed, from N-terminus to C-terminus, of the mutant Pre-F protein, a linker, and the ferritin mutant.

Preferably, the fusion protein encoded by the mRNA may further include a tag, which is a polypeptide or protein fused with the target protein using DNA recombination technology to facilitate the expression, detection, tracing, and/or purification of target protein. The tag may include, but is not limited to, Flag tag, His tag, MBP tag, HA tag, myc tag, GST tag, and/or SUMO tag.

Preferably, the mRNA includes any one of the following:
(H1) Part or all of the mRNA shown in SEQ ID No. 30-33;
(H2) A degenerate or complementary sequence of any one in (H1);
(H3) An mRNA with at least 80% identity to any one in (H1) or (H2) and encoding a protein with the same function.

The coding regions corresponding to SEQ ID No. 30-33 are shown in Table 8.

TABLE 8
Coding Regions Corresponding to SEQ ID No. 30-33

| Sequence | Coding Region of Mutant Pre-F | Coding Region of Ferritin |
|---|---|---|
| No. | Protein | Mutant |
| SEQ ID | Nucleotides 1-1422 | Nucleotides 1477-1941 |
| No. 30 | | |
| SEQ ID | Nucleotides 1-1425 | Nucleotides 1450-1944 |
| No. 31 | | |
| SEQ ID | Nucleotides 1-1422 | Nucleotides 1477-1941 |
| No. 32 | | |
| SEQ ID | Nucleotides 1-1425 | Nucleotides 1450-1944 |
| No. 33 | | |

More preferably, the first ORF includes part of the mRNA shown in SEQ ID No. 30-33. The part of mRNA shown in SEQ ID No.4-7 includes at least the coding regions of the mutant Pre-F protein and/or the coding regions of the ferritin mutant as shown in Table 8.

Alternatively, more preferably, the first ORF includes the mRNA encoding the mutant Pre-F protein in SEQ ID No. 30-33 or the sequences defined in (C2) and (C3), and the second ORF includes the mRNA encoding the ferritin mutant in SEQ ID No. 30-33 or the sequences defined in (C2) and (C3).

Further preferably, the first ORF and/or second ORF includes mRNA encoding a tag protein.

The tag may bind the proteins expressed by the first ORF and/or second ORF. The immunogenic portion and the monomeric subunit protein bind to form nanoparticles, promoting multivalent display of antigens on the nanoparticles.

The tag is preferably a motif tag.

More preferably, the motif tag protein is SpyTag and/or SpyCatcher.

Preferably, the monomeric subunit protein includes monomeric encapsulin protein, monomeric 03-33 protein, monomeric sulfur oxygenase reductase protein, monomeric dihydropteridine synthase protein, and/or monomeric pyruvate dehydrogenase complex dihydrolipoamide acetyltransferase protein.

Preferably, the mRNA includes, in addition to the coding region, a 5′ cap structure, 5′ UTR, 3′ UTR, and/or poly (A) tail.

Preferably, the mRNA sequence is natural or modified RNA, where the modified RNA includes RNA in which natural uridine is partially or fully replaced by modified uridine.

Those skilled in the art can easily mutate the nucleotide sequences encoding the aforementioned proteins or fusion proteins using known methods, such as directed evolution or site-directed mutagenesis. Nucleotide sequences with at least 75% identity to the sequences encoding the aforementioned proteins or fusion proteins, provided they encode the same functional proteins or fusion proteins, are considered derivatives of the nucleotide sequences of this invention and are equivalent to nucleotide sequences of this invention.

“Identity” refers to sequence similarity to the natural amino acid or nucleic acid sequence. “Identity” includes nucleotide sequences with at least 75%, 80%, 85%, 90%, or 95% identity to the nucleotide sequences encoding the proteins shown in SEQ ID No. 2-9 or 12-19. Identity can be assessed visually or using computer software. With computer software, identity between two or more sequences can be expressed as a percentage (%) to evaluate related sequences.

The aforementioned at least 75% identity may be at least 80%, 85%, 90% or 95% identity.

In the aforementioned biological materials, the “expression cassette” refers to DNA capable of expressing the aforementioned protein or fusion protein in host cells. This DNA may include not only a promoter to initiate transcription of the protein or fusion protein coding sequence but also a terminator to terminate transcription. Further, the expression cassette may also include an enhancer sequence.

In the aforementioned biological materials, the “vector” may be a plasmid, cosmid, phage, or viral vector.

In the aforementioned biological materials, the “microorganism” may be yeast, bacteria, algae, or fungi.

In the aforementioned biological materials, the “cell” may be a prokaryotic or eukaryotic cell.

To achieve the above objective, the invention also provides a method for preparing the aforementioned protein or fusion protein.

The method for preparing the aforementioned protein or fusion protein includes the following steps: expressing the nucleic acid molecule encoding the aforementioned protein or fusion protein in a biological organism or cell to obtain the fusion protein.

Further, the method includes the following steps: introducing the nucleic acid molecule encoding the protein or fusion protein into CHO K1Q cells to obtain recombinant cells; culturing the recombinant cells to obtain the protein or fusion protein.

Further, the nucleic acid molecule encoding the protein or fusion protein is introduced into CHO K1Q cells via a recombinant plasmid.

The recombinant plasmid is obtained by inserting the nucleic acid molecule encoding the protein or fusion protein into a vector plasmid.

In the specific embodiments of present invention, the vector plasmid is the pKS001 vector plasmid. The recombinant plasmids are recombinant plasmids pKS001-RSV-PreF-A-NP, pKS001-RSV-PreF-B-NP, pKS001-RSV-PreF-C-NP, pKS001-RSV-PreF-D-NP, pKS001-RSV-PreF-E-NP, pKS001-RSV-PreF-F-NP, pKS001-RSV-PreF-G-NP, or pKS001-RSV-PreF-H-NP.

The invention also provides a method for preparing the aforementioned biological materials. The preparation method includes designing a DNA sequence based on the encoded fusion protein and constructing a vector containing the DNA.

Preferably, the preparation method further includes transcribing and expressing mRNA from the vector.

More preferably, the invention provides a method for preparing the aforementioned mRNA. The preparation method includes designing a DNA sequence based on the monomeric subunit protein and the at least one immunogenic portion from RSV encoded by the mRNA, constructing a vector containing the DNA, and transcribing the vector to express mRNA.

Preferably, the at least one immunogenic portion from RSV includes the mutant Pre-F protein, and the monomeric subunit protein includes the ferritin mutant. The mutant Pre-F protein and the ferritin mutant are subjected to fusion expression or self-assemble.

Preferably, the mutant Pre-F protein and the ferritin mutant are subjected to fusion expression or self-assemble.

More preferably, the fusion expression is achieved via a linker peptide, or the self-assembly is achieved via a tag.

Further preferably, the tag is Spy and Catcher. In a specific embodiment, the sequences of Spy and Catcher are as shown in SEQ ID No. 28 and 29, respectively.

The invention also provides new uses for the aforementioned protein, fusion protein, biological materials, or the protein or fusion protein prepared by the aforementioned methods.

The invention provides the use of the aforementioned protein, fusion protein, biological materials, or the protein or fusion protein prepared by the aforementioned methods in any of the following (Y1-Y4):
Y1) As an immunogen;
Y2) For preparing products against respiratory syncytial virus;
Y3) For preparing products to prevent and/or treat respiratory syncytial virus infection;
Y4) For preparing products to prevent and/or treat diseases caused by respiratory syncytial virus.

To achieve the above objective, the invention further provides a vaccine.

The active ingredient of the vaccine provided by the invention is the aforementioned protein, fusion protein, biological materials, or the protein or fusion protein prepared by the aforementioned methods.

Preferably, the biological materials include mRNA.

Preferably, the vaccine is a fusion protein vaccine.

Preferably, the vaccine is an mRNA vaccine.

Further, the vaccine may further include an adjuvant.

Further, the adjuvant may be an aluminum adjuvant. The aluminum adjuvant may be one or more of aluminum hydroxide or aluminum phosphate.

Further, the mass ratio of the protein to the aluminum adjuvant may be 1:(0.5-300), or any value or range therein, such as 1:(1-275), 1:(10-200), 1:(30-180), 1:(40-100), 1:(10-80), 1:(25-75), 1:(50-75), or 1:50, etc.

Preferably, the vaccine includes dual adjuvants, comprising the aforementioned aluminum adjuvant and a CpG adjuvant.

In some embodiments, the CpG adjuvant includes any CpG adjuvant, such as CpG1018 or CpG-cjx, etc.

In some embodiments, the mass ratio of the protein to the CpG adjuvant may be 1:(0.5-300), or any value or range therein, such as 1:(1-275), 1:(10-200), 1:(30-180), 1:(40-100), 1:(10-80), 1:(25-75), 1: (50-75), or 1:50, etc.

In some embodiments, the mass ratio of the aluminum adjuvant to the CpG adjuvant in the dual adjuvants is (1-5):(5-1), or any value or range therein, such as 1:2, 1:1, or 2:1, etc.

In some embodiments, the mass ratio of the protein: aluminum adjuvant: CpG adjuvant is 1:50:50.

Further, the mRNA vaccine includes the aforementioned mRNA and lipid nanoparticles (LNPs), with the mRNA encapsulated in the LNPs.

The term “lipid nanoparticles” (LNPs) refers to particles having at least one dimension on the order of nanometers, which includes at least one lipid.

More preferably, the LNPs include a cationic lipid, neutral phospholipid, sterol lipid, and PEG-lipid.

The term “neutral phospholipid” refers to a phospholipid molecule that is uncharged and non-phosphoglyceride.

The term “PEG-lipid” refers to a molecule comprising a lipid portion and a polyethylene glycol (PEG) portion.

Preferably, the cationic lipid compound structure is selected from DLin-MC3-DMA, ALC-0315, or SM-102.

Preferably, the neutral phospholipid is selected from the group consisting of 1,2-distearoyl-sn-glycero-3-phosphocholine (DSPC), 1,2-dipalmitoyl-sn-glycero-3-phosphocholine (DPPC), 1,2-dioleoyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), 1,2-dioleoyl-sn-glycero-3-phospho-(1′-rac-glycerol) (DOPG), 1-palmitoyl-2-oleoyl-sn-glycero-3-phosphocholine (POPC), or 1-palmitoyl-2-oleoyl-sn-glycero-3-phosphoethanolamine (POPE), or a combination thereof. DSPC is preferred.

Preferably, the steroidal lipid is selected from the group consisting of avenasterol, β-sitosterol, campesterol, ergocalciferol, brassicasterol, cholestanol, cholesterol, coprostanol, dehydrocholesterol, desmosterol, dihydroergocalciferol, dihydrocholesterol, dihydroergosterol, fucosterol, dinosterol, epicholestanol, ergosterol, fucosterol, lumisterol, hydroxylated cholesterol, or cholesterol modified by polypeptides; or lanosterol, lumisterol, saringosterol, sitostanol, sitosterol, stigmastanol, stigmasterol, cholic acid, glycocholic acid, taurocholic acid, deoxycholic acid, or lithocholic acid, or a combination thereof. Cholesterol is preferred.

Preferably, the PEG-lipid is selected from 2-[(polyethylene glycol)-2000]-N,N-ditetradecylacetamide (ALC-0159), 1,2-dimyristoyl-sn-glycero-methoxypolyethylene glycol (PEG-DMG), 1,2-distearoyl-sn-glycero-3-phosphoethanolamine-N-[amino(polyethylene glycol)] (PEG-DSPE), PEG-distearoyl glycerol (PEG-DSG), PEG-dipalmitoleoyl, PEG-dioleoyl, PEG-distearoyl, PEG-diacylglyceramide (PEG-DAG), PEG-dipalmitoylphosphatidylethanolamine (PEG-DPPE), PEG-1,2-dimyristoyloxypropyl-3-amine (PEG-c-DMA), or DMG-PEG2000, or a combination thereof. DMG-PEG2000 is preferred.

Preferably, in the LNPs, the molar ratio of cationic lipid: neutral phospholipid: sterol lipid: PEG-lipid is (40-50):(5-15): (35-45):(1-5). More preferably, the ratio is 45:10:43:2.

Preferably, the mass ratio of the mRNA solution to LNPs is (2-8):1. More preferably, it is 3:1.

More preferably, the vaccine is a liquid formulation or a lyophilized powder. More preferably, the vaccine is an oral formulation, intramuscular injection formulation, intravenous injection formulation, or inhalation formulation. Further preferably, the vaccine is a nebulized inhalant or dry powder inhalant.

The vaccine of the invention may further include pharmaceutically acceptable excipients. The pharmaceutically acceptable excipients may include carriers, diluents, adjuvants or nucleotide sequences encoding adjuvants, solubilizers, binders, lubricants, suspending agents, transfection promoters, etc.

The transfection promoters include, but are not limited to, surfactants such as immunostimulatory complexes, Freund's incomplete adjuvant, LPS analogs (e.g., monophosphoryl lipid A), muramyl peptides, quinone analogs, squalene, hyaluronic acid, lipids, calcium ions, viral proteins, cations, polycations (e.g., poly-L-glutamic acid (LGS)), nanoparticles, or other known transfection promoters.

The nucleotide sequences encoding adjuvants include sequences encoding at least one of the following adjuvants: GM-CSF, IL-17, IFN-γ, IL-15, IL-21, anti-PD1/2, lactoferrin, protamine, IL-1, IL-2, IL-3, IL-4, IL-5, IL-6, IL-7, IL-8, IL-9, IL-10, IL-12, IFN-α, IFN-γ, lymphotoxin-α, hGH, MCP-1, MIP-1α, MIP-1β, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, CD40, CD40L, vascular growth factor, fibroblast growth factor, nerve growth factor, vascular endothelial growth factor, Apo-1, p55, WSL-1, DR3, TRAMP, Apo-3, AIR, LARD, NGRF, DR4, DR5, KILLER, TRAIL-R2, TRICK2, DR6, caspase ICE, Fos, c-jun, Sp-1, Ap-1, Ap-2, p38, p65Rel, MyD88, IRAK, TRAF6, IκB, inactive NIK, SAPK, SAP-1, JNK, NF-κB, Bax, TRAIL, TRAILrec, TRAILrecDRC5, TRAIL-R3, TRAIL-R4, RANK, RANK ligand, O×40, O×40 ligand, NKG2D, MICA, MICB, NKG2A, NKG2B, NKG2C, NKG2E, NKG2F, TAP1, TAP2, or functional fragments thereof.

The use of the aforementioned vaccine in any of the following Y1)-Y3) is also within the scope of protection of present invention:
Y1) For preparing products against respiratory syncytial virus;
Y2) For preparing products to prevent and/or treat respiratory syncytial virus infection;
Y3) For preparing products to prevent and/or treat diseases caused by respiratory syncytial virus.

In any of the aforementioned use or products or methods, the product may be a vaccine.

In another aspect, the invention provides a pharmaceutical composition comprising the aforementioned fusion protein, biological materials, or mRNA vaccine.

Preferably, the pharmaceutical composition is used to prevent respiratory syncytial virus infection-related diseases.

In another aspect, the invention provides a method for preparing the aforementioned pharmaceutical composition. The preparation method includes steps for preparing the fusion protein, biological materials, or mRNA.

Preferably, the preparation method includes dissolving LNPs in a solvent, mixing with mRNA, encapsulating, and preparing the mRNA pharmaceutical composition.

The LNPs are as defined above.

In another aspect, the invention provides a method for inducing an immune response. The method may include administering the aforementioned pharmaceutical composition to a subject.

In another aspect, the invention provides a method for preventing and/or treating RSV infection-related diseases. The method may include administering the pharmaceutical composition to a subject.

In the aforementioned methods, the RSV infection-related diseases may include infections of the respiratory system, digestive system, cardiovascular system, and/or nervous system.

In the aforementioned methods, the respiratory system infections may include respiratory tract infection and/or lung infection.

In the aforementioned methods, respiratory tract infections may include severe acute respiratory syndrome, hypoxic respiratory failure, sepsis, septic shock, nasopharyngitis, rhinitis, pharyngitis, tracheitis, and/or bronchitis. Lung infections may include pneumonia and/or lung injury.

In the aforementioned methods, digestive system infections may include intestinal diseases, anorexia, nausea, vomiting, abdominal pain, and/or diarrhea.

The aforementioned methods comprise administering to the subject the pharmaceutical composition, thereby inducing an immune response against RSV in the subject. The immune response may be a cellular immune response, a humoral immune response, or both.

The cellular immune response may include B-cell and T-cell immune responses.

The subject may be a human or non-human animal.

Further, the non-human animal may be a non-human mammal.

The non-human mammal may be a mouse, rat, guinea pig, hamster, pig, dog, sheep, monkey, rabbit, cat, cow, or horse, but is not limited thereto.

The subject disclosed here includes, but is not limited to, healthy subjects, symptomatic infected subjects, asymptomatic infected subjects, or recovered subjects (subjects who have recovered from infection).

Administration disclosed here includes, but is not limited to, intramuscular injection, subcutaneous injection, intradermal injection, intravenous injection, intra-arterial injection, intraperitoneal injection, microneedle injection, mucosal administration, oral administration, oronasal spray, or nebulized inhalation.

Advantages of the Invention:
1. The invention enhances the immunogenicity, stability, and safety of the Pre-F protein through specific antigen mutation designs and further improves immunogenicity by displaying the desired epitopes on the surface of nanoparticles. Experiments demonstrate that the vaccine prepared by the invention achieves excellent immune effects at low doses, with the RSV-PreF-C-NP group achieving a neutralizing titer of 19,836.
2. The invention addresses the poor stability of wild-type antigens. The ferritin-PreF fusion protein prepared by the invention can induce neutralizing antibodies against RSV in vivo, providing the organism with corresponding immune protection.
3. The ferritin-PreF fusion protein prepared by the invention effectively activates the cellular immune mechanism of the organism, inducing a balanced Th1/Th2 immune response, thereby avoiding excessive immune reactions caused by Th2 bias and ensuring good safety.
4. The mRNA vaccine of the invention can induce neutralizing antibodies against RSV in vivo, providing humoral immune protection. Additionally, the mRNA vaccine effectively activates the cellular immune mechanism, increasing the frequency of IFN-γ secretion by splenocytes and inducing cellular immunity, thereby providing longer-lasting and more comprehensive protection.
5. Through screening of adjuvant, the invention identifies suitable adjuvant combinations and effective concentration ranges. Dual adjuvants exhibit synergistic effects, significantly improving the immune efficacy of the RSV vaccine.

The invention obtains ferritin-PreF fusion protein nanoparticles displaying multiple Pre-F antigens densely arrayed on the surface, by mutating the RSV Pre-F sequence and ferritin nanoparticles and expressing the mutant Pre-F protein and ferritin mutant as a fusion protein in eukaryotic cells. The resulting ferritin-PreF fusion protein nanoparticles stabilize and expose desired antigenic epitopes and disrupt or mask undesired epitopes, thereby effectively enhancing immunogenicity, production stability, and safety. Experiments show that injecting the ferritin-PreF fusion protein into mice produces high-titer protective serum with strong neutralizing activity against live viruses. Stability and safety tests confirm that the ferritin-PreF fusion protein exhibits sufficient physical stability and excellent safety. The mRNA vaccine effectively activates the cellular immune mechanism, increasing the frequency of IFN-γ secretion by splenocytes and inducing cellular immunity for longer-lasting and more comprehensive protection.

### BRIEF DESCRIPTION OF THE DRAWINGS

FIG. 1: SDS-PAGE electrophoresis detection results of ferritin-PreF fusion protein.

A: Lane 1 shows the elution product of purified RSV-PreF-A-NP; lanes 2 and 3 show the flow-through and supernatant, respectively; lane 4 shows the molecular weight marker (Solarbio, Cat. No. PR1910, same below).

B: Lane 1 shows the elution product of purified RSV-PreF-B-NP; lanes 2 and 3 show the flow-through and molecular weight marker, respectively.

C: Lane 1 shows the flow-through of purified RSV-PreF-C-NP; lane 2 shows the elution product of purified RSV-PreF-C-NP; lanes 3 and 4 show the elution products of purified RSV-PreF-D-NP and RSV-PreF-E-NP, respectively.

D: Lane 1 shows the elution product of purified RSV-PreF-F-NP; lane 2 shows the molecular weight marker.

E: Lanes 1 and 2 show the flow-through and elution product of purified RSV-PreF-G-NP, respectively.

F: Lane 1 shows the molecular weight marker; lane 2 shows the flow-through of purified RSV-PreF-H-NP; lane 3 is a blank lane; lane 4 shows the elution product of purified RSV-PreF-H-NP.

FIG. 2: Western blot (WB) detection results of purified ferritin-PreF fusion protein. From left to right: WB results of purified RSV-PreF-A-NP, RSV-PreF-B-NP, RSV-PreF-C-NP, RSV-PreF-D-NP, RSV-PreF-E-NP, RSV-PreF-F-NP, RSV-PreF-G-NP, and RSV-PreF-H-NP.

FIG. 3: Nanoparticle morphology of ferritin-PreF fusion protein.

A-H: Electron micrographs of purified RSV-PreF-A-NP, RSV-PreF-B-NP, RSV-PreF-C-NP, RSV-PreF-D-NP, RSV-PreF-E-NP, RSV-PreF-F-NP, RSV-PreF-G-NP, and RSV-PreF-H-NP, respectively.

FIG. 4: Immunogenicity study results of ferritin-PreF fusion protein vaccine.

FIG. 5: Log2 values of neutralizing titers in mice immunized with ferritin-PreF fusion protein.

FIG. 6: ELISA titers of IgG1 and IgG2a in serum of mice immunized with ferritin-PreF fusion protein RSV-PreF-C-NP, compared with historical formalin-inactivated vaccine controls.

FIG. 7: Frequency of IFN-γ-secreting splenocytes after stimulation with different mRNA vaccines.

### EXAMPLES

Hereinafter, the invention will be explained in more detail through specific embodiments, which are provided to illustrate the invention but not to limit its scope. The examples may serve as guidelines for further modifications by those skilled in the art, but do not limit this invention in any way.

Experimental methods in the following examples are conventional unless otherwise specified, performed according to techniques or conditions described in the literature or product manuals. Materials and reagents used are commercially available or prepared by known methods unless otherwise specified.

The RSV virus A-type Long strain in the following examples is recorded in the literature: “Cultures of HEp-2 cells persistently infected by human respiratory syncytial virus differ in chemokine expression and resistance to apoptosis as compared to lytic infections of the same cell type”.

The RSV virus B-type BA9 strain in the following examples is recorded in the literature: “Genetic Diversity and Molecular Epidemiology of Circulating Respiratory Syncytial Virus in Central Taiwan,2008-2017.”

The similar vaccine developed by Johnson & Johnson in the following examples is recorded in the literature: “preF immunogenicity and protective efficacy of adenoviral and subunit RSVvaccines based on stabilized prefusion F protein in preclinical models.”

The formalin-inactivated vaccine FI-RSV in the following examples is documented in the literature: “Enhanced pulmonary histopathology induced by respiratory syncytial virus (RSV) challenge of formalin-inactivated RSV-immunized BALB/c mice is abrogated by depletion of interleukin-4 (IL-4) and IL-10.”

### Example 1. Design, Preparation and Purification of Ferritin-PreF Fusion Protein

The Pre-F-related sequence of RSV was mutated and designed to obtain Pre-F mutant proteins, which were then fused with ferritin-related sequences to form ferritin-PreF subunit complexes. Through utilization of ferritin's self-assembly properties, nanoparticles with effective Pre-F antigen presentation were formed. The specific procedures are described below:

### I. Design of Ferritin-PreF Fusion Protein

### 1. Design of RSV Pre-F-Related Sequence

To expose and stabilize desired epitopes while disrupting or masking undesired epitopes, the amino acid sequence of RSV Pre-F protein (shown as SEQ ID No. 1) was modified with at least one of the following mutations 1)-18) to generate Pre-F mutant proteins:
1) Isoleucine (I) at position 67 was mutated to asparagine (N).
2) Serine(S) at position 88 was mutated to asparagine (N).
3) Cysteine (C) at position 110 was mutated to alanine (A).
4) Asparagine (N) at position 144 was mutated to glycine (G), with cysteine (C) inserted between positions 144 and 145.
5) Tyrosine (Y) at position 159 was mutated to cysteine (C).
6) Cysteine (C) at position 173 was deleted.
7) Alanine (A) at position 202 was mutated to cysteine (C).
8) Isoleucine (I) at position 227 was mutated to asparagine (N).
9) Serine(S) at position 236 was mutated to arginine (R).
10) Serine(S) at position 248 was mutated to cysteine (C).
11) Glutamic acid (E) at position 289 was mutated to asparagine (N).
12) Serine(S) at position 309 was mutated to asparagine (N).
13) Arginine (R) at position 334 was mutated to tyrosine (Y).
14) Asparagine (N) at position 344 was mutated to glutamic acid (E).
15) Serine(S) at position 370 was mutated to glycine (G).
16) Asparagine (N) at position 389 was mutated to cysteine (C).
17) Cysteine (C) at position 419 was mutated to tyrosine (Y).
18) Arginine (R) at position 468 was mutated to asparagine (N).

The resulting Pre-F mutant proteins were designated as RSV-PreF-A, RSV-PreF-B, RSV-PreF-C, RSV-PreF-D, RSV-PreF-E, RSV-PreF-F, RSV-PreF-G and RSV-PreF-H.

The amino acid sequences of Pre-F mutant proteins RSV-PreF-A, RSV-PreF-B, RSV-PreF-C, RSV-PreF-D, RSV-PreF-E, RSV-PreF-F, RSV-PreF-G and RSV-PreF-H were shown in SEQ ID No. 2-SEQ ID No. 9 respectively.

### 2. Design of Nanoparticle Sequence

To enhance particle stability and integrity, the amino acid sequence (SEQ ID No. 10) of ferritin was modified with at least one of the following mutations 1)-3) to obtain ferritin mutant:
1) Asparagine (N) at position 15 was mutated to glutamine (Q).
2) Serine(S) at position 96 was mutated to asparagine (N).
3) Tyrosine (Y) at position 119 was mutated to arginine (R).

In present invention, the amino acid sequence of the ferritin mutant was shown in SEQ ID No. 11.

### II. Preparation of Ferritin-PreF Fusion Protein

### 1. Construction of Recombinant Plasmids

### 1) Gene Fusion Design of Ferritin-PreF

The Pre-F mutant proteins and ferritin mutant were fused via a linker (SGSGGGSG) (SEQ ID NO.34) to prepare ferritin-PreF fusion proteins. Each fusion protein was constructed from N- to C-terminus as: Pre-F mutant protein—linker (SGSGGGSG) (SEQ ID NO.34)—ferritin mutant.

The resulting ferritin-PreF fusion proteins were designated as RSV-PreF-A-NP, RSV-PreF-B-NP, RSV-PreF-C-NP, RSV-PreF-D-NP, RSV-PreF-E-NP, RSV-PreF-F-NP, RSV-PreF-G-NP and RSV-PreF-H-NP, with their amino acid sequences shown in SEQ ID No. 12-SEQ ID No. 19, and coding gene sequences shown in SEQ ID No. 20-SEQ ID No. 27, respectively.

Plasmids containing the coding gene sequences encoding the aforementioned each ferritin-PreF fusion protein were synthesized by GenScript Biotech (Nanjing) and designated as plasmid RSV-PreF-A-NP, RSV-PreF-B-NP, RSV-PreF-C-NP, RSV-PreF-D-NP, RSV-PreF-E-NP, RSV-PreF-F-NP, RSV-PreF-G-NP and RSV-PreF-H-NP.

### 2) Construction of Recombinant Plasmids

The vector plasmid pKS001 (Zhongshan Kangtiancheng Biotech, Cat #A14101) was digested with restriction enzymes Hind III-HF and Not I-HF (NEB, Cat #R3104V and R3189L) to obtain the backbone vector.

Plasmids RSV-PreF-A-NP, RSV-PreF-B-NP, RSV-PreF-C-NP, RSV-PreF-D-NP, RSV-PreF-E-NP, RSV-PreF-F-NP, RSV-PreF-G-NP and RSV-PreF-H-NP were digested with Hind III-HF and Not I-HF respectively, to obtain the target fragment respectively.

The backbone vector and target fragments were ligated using Quick Ligase (NEB, Cat # M2200L), then transformed into E. coli Trans10 competent cells (TransGen Biotech, Cat #CD101). Positive clones were screened, and plasmids were extracted and verified by sequencing. The plasmids confirmed by sequencing were designated as recombinant plasmids pKS001-RSV-PreF-A-NP, pKS001-RSV-PreF-B-NP, pKS001-RSV-PreF-C-NP, pKS001-RSV-PreF-D-NP, pKS001-RSV-PreF-E-NP, pKS001-RSV-PreF-F-NP, pKS001-RSV-PreF-G-NP and pKS001-RSV-PreF-H-NP.

Sequencing results confirmed that these recombinant plasmids pKS001-RSV-PreF-A-NP, pKS001-RSV-PreF-B-NP, pKS001-RSV-PreF-C-NP, pKS001-RSV-PreF-D-NP, pKS001-RSV-PreF-E-NP, pKS001-RSV-PreF-F-NP, pKS001-RSV-PreF-G-NP and pKS001-RSV-PreF-H-NP were respectively obtained by replacing the DNA fragment between Hind III-HF and Not I-HF sites in pKS001 vector with the DNA molecules shown in SEQ ID No.20, SEQ ID No.21, SEQ ID No.22, SEQ ID No.23, SEQ ID No.24, SEQ ID No.25, SEQ ID No.26 and SEQ ID No.27, while maintaining all other sequences of the pKS001 vector, respectively.

### 2. Expression of Ferritin-PreF Fusion Protein

The aforementioned recombinant plasmids pKS001-RSV-PreF-A-NP, pKS001-RSV-PreF-B-NP, pKS001-RSV-PreF-C-NP, pKS001-RSV-PreF-D-NP, pKS001-RSV-PreF-E-NP, pKS001-RSV-PreF-F-NP, pKS001-RSV-PreF-G-NP and pKS001-RSV-PreF-H-NP were respectively electroporated into CHO K1Q cells (Kangcheng Biopharma, Cat #A14101), and expressed in cells, with high-expression cell lines selected.

Electroporation was carried out with the EBXP-F1 electroporator (Suzhou Yida Biotechnology Co., Ltd.), following these steps:
1) Pre-electroporation preparation: Buffer, cell culture medium, and D-PBS were equilibrated to room temperature 30 minutes before electroporation.
2) Cell collection and counting: Cells were suspended uniformly, placed in a centrifuge tube, and counted.
3) Centrifugation: The required cell suspension was transferred into a new centrifuge tube, and centrifuged at 1,000 rpm for 5 minutes (Suzhou Guofei Lab Instruments, Cat No.: TDL-5A).
4) D-PBS wash: Supernatant was removed, and cells were resuspended in 1 mL D-PBS (Thermo Fisher Gibco, Cat #2334304) followed by centrifugation at 1,000 rpm for 5 minutes.
5) DNA-cell-buffer mixture: After removing D-PBS, electroporation buffer (Suzhou Yida Biotech, Cat #H10305) and 10 ug plasmid were added and mixed gently.
6) Electro transformation: The cell-plasmid suspension (200 μL+DNA volume per cuvette) was transferred to H1 electroporation cuvettes (Suzhou Yida Biotech, Cat #H10201). The electroporation cuvette was placed into the holder, and the cells were electroporated under conditions specified in Table 1.

TABLE 1
Electroporation Conditions

| | | Pulse | | Pulse | Buffer | | | | |
|---|---|---|---|---|---|---|---|---|---|
| Cell | Voltage | Width | Pulse | Interval | Volume | | | | |
| Number | (V) | (us) | Number | (ms) | (ul) | Buffer | Plasmid | Cuvette | Electroporator |
| 1 × 10⁷ | 200 | 2000 | 6 | 1000 | 200 | EBEL | 10 ug | Yida | H1 |
| | | | | | | | | 2 mm | |

7) Culture: Electroporated cells were transferred to T25 flasks (NEST Biotechnology, Cat # 707003) containing 10 mL CDO4 medium (Kangcheng Biopharma, Cat #A11004) and cultured for 48 hours.

The steps for culture and screening of cell clones are as follows: Cells in above T25 flasks were sampled, and viability was monitored using cell counter (Countess II FL, Sanofi). At >70% viability, cells were plated in 96-well plates at 10,000 cells/well, and cultured in CD04 medium containing 25 mM MSX (Sigma, Cat #M5379-1G). Positive clones were selected by ELISA and scaled up to 125 mL shake flasks (Wuxi Naisi Biotechnology, Cat #781011). After 5-7 days, when viability dropped to 50-80%, supernatant was collected for ELISA.

ELISA method: The supernatant was subjected to 10-fold, 100-fold, 1000-fold, 10000-fold dilution, and coated. 1500-fold diluted F protein antibody (PujianBiotech (Wuhan), Cat #62814) was used as primary antibody. Goat anti-human IgG-HRP (Solarbio, Cat #SE101-1ml) was used as secondary antibody. Signals were read using a microplate reader (Shanghai Kehua, Cat #RD-SH-012). The sample showing the strongest signal was chosen as the highest-expression one. The supernatant from the highest-expressing sample was collected for purification.

### III. Purification of Ferritin-PreF Fusion Protein

The cell culture supernatant was purified following the method described in “Flexible RSV Prefusogenic Fusion Glycoprotein Exposes Multiple Neutralizing Epitopes that May Collectively Contribute to Protective Immunity”, using Capto Lentil Lectin (Cytiva, Cat #17548902), Q Sepharose FF (Cytiva, Cat #17051060), Capto Core 400 (Cytiva, Cat #17372402), Superose 6 prep grade (Cytiva, Cat #10321079). The specific purification steps are as follows:

Cell culture supernatant was centrifuged at 8,000 rpm for 20 minutes and filtered through 0.45 μm membranes (Jinteng, Cat #JTSF 025013/014), thereby obtaining about 100 mL solution. The 100 mL solution was adjusted to 200 mL with equilibration buffer. The QFF column was equilibrated with equilibration buffer. Samples were loaded via pump A1, at a flow rate of 1.5 mL/min. After loading, the column was washed with equilibration buffer until the absorbance returned to the pre-loading baseline level and stabilized. Gradient elution was performed with elution buffer (20 mM Tris, 0.5 M NaCl, pH 8.5) at a flow rate of 2 mL/min, 0-100% B, 50 minutes. Elution peaks were collected. The supernatant was concentrated 5-10 fold, then loaded onto a Superose 6 prep grade column at 1 mL/min. Peak fractions were collected to obtain the ferritin-PreF fusion protein solution, which was concentrated for SDS-PAGE and Western blot analysis.

SDS-PAGE Analysis: 80 μL ferritin-PreF fusion protein solution was mixed with 20 μL 5× loading buffer. After heating at 95° C. for 10 minutes and centrifugation, 15 μL supernatant was analyzed by SDS-PAGE. Staining was performed to observe protein expression.

### Western Blot Analysis

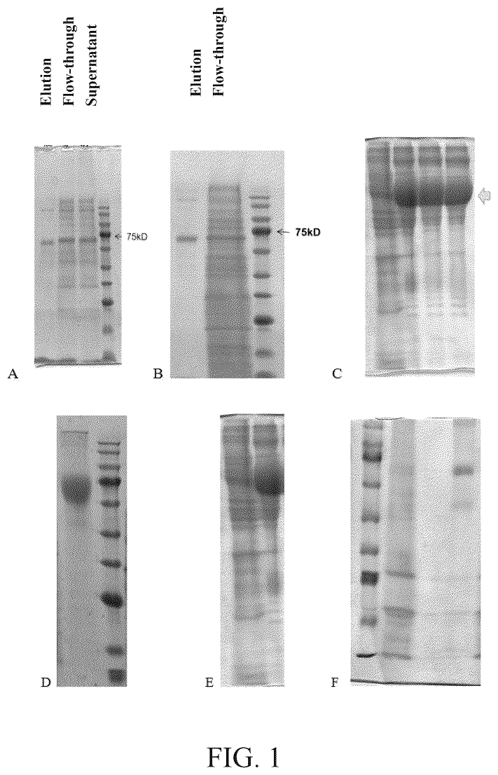

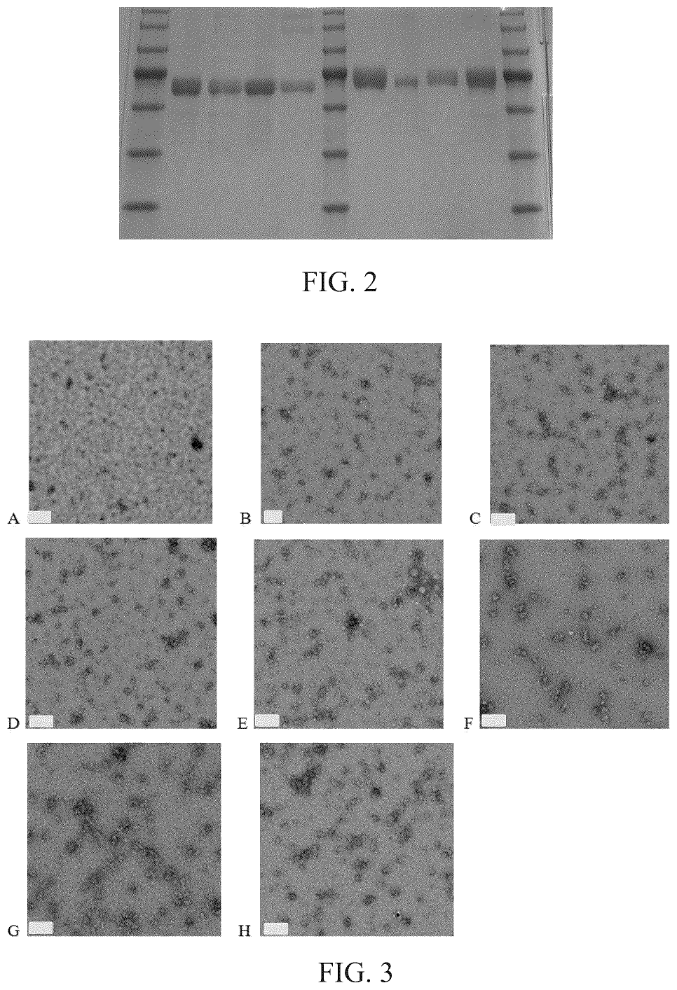

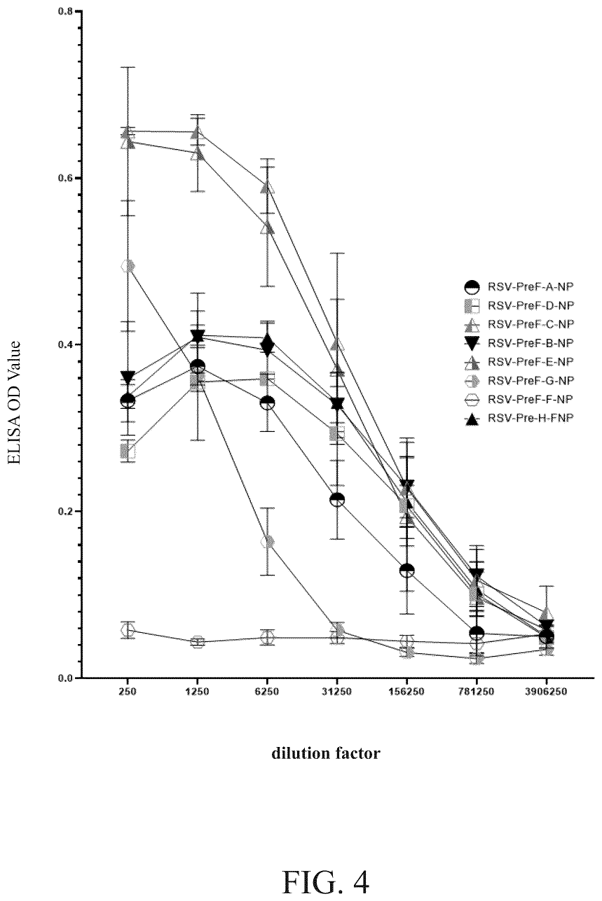

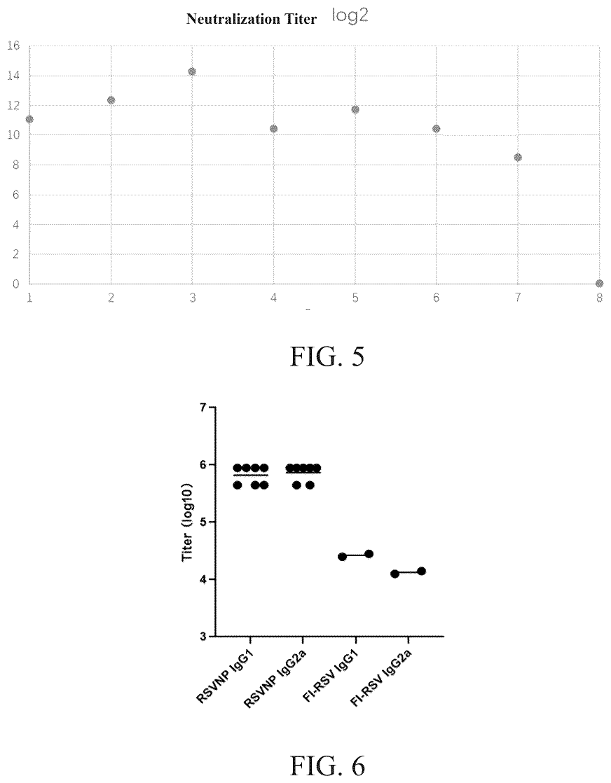

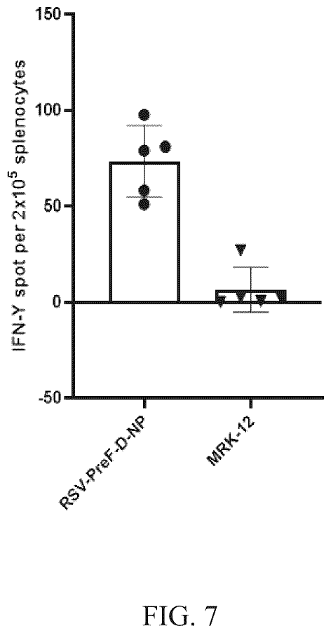

1. SDS-PAGE electrophoresis: 10% SDS-PAGE gel with 1.0 mm thickness was prepared. Electrophoresis was performed in 1× SDS running buffer. 20 μL protein sample was loaded and run at 80V until entering separation gel, then switched to 130V.
2. Semi-dry transfer: The transfer tank (Junyi, Cat #JY-ZY3) was used. PVDF membrane and 6 filter papers were soaked in 1× transfer buffer (39 mM glycine, 48 mM Tris, 0.037% SDS, 20% methanol). After electrophoresis, excess gel was removed. A graphite electrode-transfer membrane gel complex was assembled in the order of: anode electrode-three layers of wet filter paper—PVDF membrane—protein gel—three layers of wet filter paper—cathode electrode. After power was applied, transfer was performed at 1.0 mA/cm² for 60 minutes.
3. Blocking: Membrane was blocked with 5% skim milk in PBST at 37° C. for 1 hour.
4. Primary antibody incubation: The blocked membrane was immersed in 1×PBST buffer containing primary antibody (Invitrogen, RSV Fusion Protein Polyclonal Antibody, Cat #XD3556234B), and incubated at 37° C. for 60 minutes. After incubation, the membrane was washed three times with 1×PBST on a shaker at 70 rpm, 10 minutes each time.
5. Secondary antibody incubation: Diluted secondary antibody (Bioworld, Goat Anti-Rabbit IgG (H+L) HRP, Cat #AA092030) in 1×PBST was added, and incubated at 37° C. for 45 minutes. After incubation, the membrane was washed three times with 1×PBST on a shaker at 70 rpm, 10 minutes each time.
6. Detection: DAB substrate kit (Solarbio, Cat #DA1016) was used for color development. The results of SDS-PAGE was shown in FIG. 1 and the results of Western blot was shown in FIG. 2. The results confirmed that, the recombinant plasmids pKS001-RSV-PreF-A-NP. pKS001-RSV-PreF-B-NP, pKS001-RSV-PreF-C-NP, pKS001-RSV-PreF-D-NP, pKS001-RSV-PreF-E-NP, pKS001-RSV-PreF-F-NP, pKS001-RSV-PreF-G-NP and pKS001-RSV-PreF-H-NP were successfully expressed in CHO K1Q cells, thereby obtaining target protein with an approximate size of 74 kDa respectively. The target proteins are ferritin-PreF fusion RSV-PreF-A-NP, RSV-PreF-B-NP. RSV-PreF-C-NP. RSV-PreF-D-NP, RSV-PreF-E-NP. RSV-PreF-F-NP, RSV-PreF-G-NP and RSV-PreF-H-NP.

### Example 2. Nanoparticle Morphology Analysis of Ferritin-PreF Fusion Proteins

The purified products of ferritin-PreF fusion proteins RSV-PreF-A-NP, RSV-PreF-B-NP, RSV-PreF-C-NP, RSV-PreF-D-NP, RSV-PreF-E-NP, RSV-PreF-F-NP, RSV-PreF-G-NP, and RSV-PreF-H-NP prepared in Example 1 were subjected to negative staining respectively. The detailed negative staining procedure was performed as follows:

The ultrathin carbon film was pre-evacuated for 3 minutes using a Harrick Basic Plasma Cleaner PDC-32G-2 instrument, followed by glow discharge at the medium setting for 30 seconds, and was removed. 4 μL sample was pipetted onto the carbon film and allowed to stand horizontally for 1 minute. Excess liquid was absorbed using filter paper. 7 μm 2% uranyl acetate was applied and left for 1 minute before being absorbed with filter paper. After drying for several minutes, the negatively stained purified samples were observed using an FEI Tecnai Arctica TEM D683 transmission electron microscope.

The results were shown in FIG. 3. The results demonstrated that well-defined nanoparticles were observed under TEM in all purified samples of ferritin-PreF fusion proteins (RSV-PreF-A-NP, RSV-PreF-B-NP, RSV-PreF-C-NP, RSV-PreF-D-NP, RSV-PreF-E-NP, RSV-PreF-F-NP, RSV-PreF-G-NP and RSV-PreF-H-NP). Transmission electron microscopy analysis revealed clear nanoparticle morphology with good structural integrity.

### Example 3. Immunogenicity Study of Ferritin-PreF Fusion Proteins

### I. Immunization

### 1. Materials and Methods

Materials: Female 6-8 weeks old Balb/c mice (SPF (Beijing) Biotechnology Co., Ltd. Cat No.: B201-02).

Methods: A total of 64 Balb/c mice (6-8 weeks old) were randomly divided into 8 groups (n=8 per group). Each group was treated as follows:

RSV-PreF-A-NP: The mice were intramuscularly injected in the thigh with 1 μg of ferritin-PreF fusion protein RSV-PreF-A-NP, 50 μg aluminum hydroxide adjuvant (Changchun Institute of Biological Products Co., Ltd., Batch No.: ZP18-003-202106), and 100 μL PBS buffer (Solarbio, Cat. No.: P1020) on days 0 and 21.

RSV-PreF-B-NP: The mice were intramuscularly injected in the thigh with 1 μg of ferritin-PreF fusion protein RSV-PreF-B-NP, 50 μg aluminum hydroxide adjuvant (Changchun Institute of Biological Products Co., Ltd., Batch No.: ZP18-003-202106), and 100 μL PBS buffer (Solarbio, Cat. No.: P1020) on days 0 and 21.

RSV-PreF-C-NP: The mice were intramuscularly injected in the thigh with 1 μg of ferritin-PreF fusion protein RSV-PreF-C-NP, 50 μg aluminum hydroxide adjuvant (Changchun Institute of Biological Products Co., Ltd., Batch No.: ZP18-003-202106), and 100 μL PBS buffer (Solarbio, Cat. No.: P1020) on days 0 and 21.

RSV-PreF-D-NP: The mice were intramuscularly injected in the thigh with 1 μg of ferritin-PreF fusion protein RSV-PreF-D-NP, 50 μg aluminum hydroxide adjuvant (Changchun Institute of Biological Products Co., Ltd., Batch No.: ZP18-003-202106), and 100 μL PBS buffer (Solarbio, Cat. No.: P1020) on days 0 and 21.

RSV-PreF-E-NP: The mice were intramuscularly injected in the thigh with 1 μg of ferritin-PreF fusion protein RSV-PreF-E-NP, 50 μg aluminum hydroxide adjuvant (Changchun Institute of Biological Products Co., Ltd., Batch No.: ZP18-003-202106), and 100 μL PBS buffer (Solarbio, Cat. No.: P1020) on days 0 and 21.

RSV-PreF-F-NP: The mice were intramuscularly injected in the thigh with 1 μg of ferritin-PreF fusion protein RSV-PreF-F-NP, 50 μg aluminum hydroxide adjuvant (Changchun Institute of Biological Products Co., Ltd., Batch No.: ZP18-003-202106), and 100 μL PBS buffer (Solarbio, Cat. No.: P1020) on days 0 and 21.

RSV-PreF-G-NP: The mice were intramuscularly injected in the thigh with 1 μg of ferritin-PreF fusion protein RSV-PreF-G-NP, 50 μg aluminum hydroxide adjuvant (Changchun Institute of Biological Products Co., Ltd., Batch No.: ZP18-003-202106), and 100 μL PBS buffer (Solarbio, Cat. No.: P1020) on days 0 and 21.

RSV-PreF-H-NP: The mice were intramuscularly injected in the thigh with 1 μg of ferritin-PreF fusion protein RSV-PreF-H-NP, 50 μg aluminum hydroxide adjuvant (Changchun Institute of Biological Products Co., Ltd., Batch No.: ZP18-003-202106), and 100 μL PBS buffer (Solarbio, Cat. No.: P1020) on days 0 and 21.

### II. Antibody Detection in Serum by ELISA

On day 28 post-immunization (approximately 6 weeks), mouse serum samples were collected for ELISA analysis. ELISA Procedure: Microplates were coated with RSV F protein (Sino Biological, Cat. No.: 11049-V08B) at 200 ng per well. Mouse serum samples were used as primary antibody, and were serially diluted 250-fold, 1,250-fold, 6,250-fold, 31,250-fold, 156,250-fold, 781,250-fold, 3,906,250-fold. Anti-mouse secondary antibody (Cell Signaling Technology, Cat. No.: 7076S) was used as the secondary antibody. Absorbance was measured using a microplate reader (Shanghai Kehua, Cat. No.: RD-SH-012).

The results of ELISA titers detected in sera from twice-immunized mice were shown in FIG. 4. The results demonstrated that, the ELISA signal remained detectable when diluted 781,250-fold for the following ferritin-PreF fusion proteins: RSV-PreF-D-NP, RSV-PreF-C-NP, RSV-PreF-B-NP, RSV-PreF-E-NP, RSV-PreF-G-NP, and RSV-PreF-H-NP. Among these, RSV-PreF-C-NP exhibited the highest ELISA titer.

### III. CPE Neutralization Assay in Mouse Serum

The TCID₅₀ of RSV type A Long strain cultured in HEp-2 cells maintained in DMEM medium containing 10% fetal bovine serum was 2.81E+07. Eight serum samples from each experimental group were diluted with DMEM containing 2% fetal bovine serum. Starting with an initial 40-fold dilution, serial 3-fold dilutions were performed to 29,160-fold. Each diluted serum sample was mixed with an equal volume of virus suspension (200 TCID50) and incubated at 37° C. for 1 hour. The serum-virus mixtures were added to Hep-2 cell plate at 200 μL/well, with three replicate wells per mouse serum. Cells were cultured at 37° C. for 5-7 days, and cytopathic effects (CPE) were recorded.

The results were shown in Table 2 and FIG. 5. The results demonstrated that, the RSV-PreF-C-NP group exhibited the highest mean neutralization titer of 19,836. The RSV-PreF-C-NP group showed a mean neutralizing titer of 14.3 (Log2). The neutralization titers (Log2) of RSV-PreF-A-NP, RSV-PreF-D-NP, RSV-PreF-C-NP, and RSV-PreF-E-NP groups each exceeded the highest Log2 titer against Strain A at week 6 post-immunization achieved by similar vaccines developed by Johnson & Johnson (The highest Log2 titer against Strain A at week 6 post-immunization achieved by similar vaccines developed by Johnson & Johnson was about 11, and the converted titer was approximately 2,100.)

TABLE 2
Mean Neutralization Titers

| No. | Group | Mean Neutralization Titer |
|---|---|---|
| 1 | RSV-PreF-A-NP | 2143 |
| 2 | RSV-PreF-D-NP | 5217 |
| 3 | RSV-PreF-C-NP | 19836 |
| 4 | RSV-PreF-B-NP | 1372 |
| 5 | RSV-PreF-E-NP | 3343 |
| 6 | RSV-PreF-G-NP | 1372 |
| 7 | RSV-PreF-F-NP | 361 |
| 8 | RSV-Pre-H-FNP | 1 |

According to the experimental method described above, the RSV A Long strain was replaced with the RSV B BA9 strain, and the same titer analysis procedure was performed using the serum samples from each group of mice.

The results were shown in Table 3. It was demonstrated that the mean neutralizing titer of mouse serum of the RSV-PreF-C-NP group was 10,568, with a log2 value of 13.4. The titers of RSV-PreF-A-NP, RSV-PreF-D-NP, RSV-PreF-C-NP, and RSV-PreF-E-NP approached or exceeded 2,100.

These results indicated that when the ferritin-PreF fusion protein prepared by the present invention is administered to mice, high-titer protective serum can be obtained. Furthermore, the mouse serum was shown to generate high neutralizing titers against both major epidemic strains of RSV A type and B type.

TABLE 3
Mean Neutralization Titers

| No. | Group | Mean Neutralization Titer |
|---|---|---|
| 1 | RSV-PreF-A-NP | 1807 |
| 2 | RSV-PreF-D-NP | 4359 |
| 3 | RSV-PreF-C-NP | 10568 |
| 4 | RSV-PreF-B-NP | 1031 |
| 5 | RSV-PreF-E-NP | 3859 |
| 6 | RSV-PreF-G-NP | 1217 |
| 7 | RSV-PreF-F-NP | 214 |
| 8 | RSV-Pre-H-FNP | 1 |

### Example 4. Stability Test of Ferritin-PreF Fusion Protein

To verify the stability of the ferritin-PreF fusion protein prepared according to the present invention, the purified ferritin-PreF fusion proteins from each group were subjected to physical stability testing (physical stress challenge). The specific procedure was as follows:

Five aliquots of the ferritin-PreF fusion protein solution (40 μg/μL) and its gradient dilutions were placed in the following conditions for 1 hour (corresponding to Columns 1-5 from left to right in the figure, respectively): pH 7.4 (25° C.), pH 3.8 (25° C.), pH 10 (25° C.), 50° C. (pH 7.4), 70° C. (pH 7.4). ELISA analysis was then performed using the same method as in the cell clone screening.

The results demonstrated that the antigen-binding activity of each group fusion protein was maintained at over 75% of the original untreated protein after exposure to different pH and temperature conditions. This indicated that all the ferritin-PreF fusion proteins prepared in the present invention possess sufficient physical stability. The stability results for the RSV-PreF-C-NP protein were presented in Table 4, with similar results observed for the other groups.

TABLE 4
Percentage of ELISA Signal Intensity Retained
After Physical Stress Challenges

| pH Treatment | Temperature |
|---|---|
| 7.4 | Treatment |

| (Untreated) | 3.8 | 10 | 50° C. | 70° C. |
|---|---|---|---|---|
| 100.00% | 77.33% | 83.62% | 91.22% | 77.08% |

Note:
Data in the table are averaged from 10-, 100-, 1000-, and 10000-fold dilution series.

### Example 5. Safety Evaluation of Ferritin-PreF Fusion Protein

To assess the Th1/Th2 balance in the immune response induced by the ferritin-PreF fusion protein prepared according to the present invention (Reference: Immunological Lessons from Respiratory Syncytial Virus Vaccine Development), ELISA analysis of IgG1 and IgG2a was performed using serum samples collected from mice immunized with the RSV-PreF-C-NP fusion protein. The ELISA assay was conducted under the same conditions as the serum ELISA detection, with the following secondary antibodies: IgG1-specific secondary antibody (abcam, Cat. No.: GR3395386-5) and IgG2a-specific secondary antibody (abcam, Cat. No.: GR3413688-1). Formalin-inactivated FI-RSV vaccine and a similar vaccine developed by Johnson & Johnson were used as controls.

The results were shown in FIG. 6. It was demonstrated that, the IgG1 and IgG2a titers in the serum reached approximately 6 (see RSVNP IgG1 and RSVNP IgG2a in FIG. 6). The IgG2a/IgG1 ratio was approximately 1, which was significantly superior to that of the formalin-inactivated vaccine (see FI-RSVNP IgG1 and FI-RSV IgG2a in FIG. 6) and even exceeded that of the Johnson & Johnson-developed similar vaccine (IgG2a ˜5.8, IgG1 ˜5). These findings indicated that the ferritin-PreF fusion protein of the present invention induced a balanced Th1/Th2 immune response upon administration, thereby avoiding vaccine-enhanced disease (VED) caused by Th2-skewed immunity. This demonstrated its favorable safety profile.

### Example 6. Self-Assembly of Ferritin and PreF Expressed Separately

Tags were respectively incorporated into the sequences of ferritin and PreF mutant protein to enable 1:1 self-assembly via tag interaction. In the present application, a Catcher tag (GAMVTTLSGLSGEQGPSGDMTTEEDSATHIKFSKRDEDGRELAGATMELRDSSGKTIST WISDGHVKDFYLYPGKYTFVETAAPDGYEVATPIEFTVNEDGQVTVDGEATEGDAHT, SEQ ID No. 28) was added to the N-terminus of ferritin through a linker, while a Spy tag (RGVPHIVMVDAYKRYK, SEQ ID No. 29) was added to the C-terminus of the PreF protein through a linker. After expression, the two components were assembled into nanoparticles via Spy-Catcher interaction.

### Construction of mRNA Plasmids

The target sequences for mRNA construction were inserted into the pVAX1 vector (provided by GenScript Biotech Corporation, Nanjing, China; plasmid map shown in the figure) using HindIII and XbaI restriction sites. The relevant plasmids were synthesized and provided by GenScript Biotech Corporation, Nanjing, China.

### Example 7. DNA Electroporation in Mice-In Vivo Screening

Immunization: Female Balb/c mice (6-8 weeks old) (Sibeifu (Beijing) Biotechnology Co., Ltd., Cat. No.: B201-02).

Experimental Method: Sixty-four Balb/c mice (6-8 weeks old) were randomly divided into 8 groups (n=8 per group). The treatment for each group was as follows:

The mice were electroporated with plasmids containing the 8 DNA sequences (SEQ ID No. 20-27). A 50 μg dose of the vector plasmid was injected into the posterior thigh muscle, followed by the application of six electrical pulses (60 V, 20 ms) using an electroporation device (Terasa Healthcare Sci-Tech, China). Mice electroporated with 50 μg of pKS plasmid were used as negative controls. Immunization was performed twice at two-week intervals. Serum samples collected after the second immunization were subjected to neutralization assays to compare neutralizing protection efficiency.

### Neutralization Assay

TCID50 of RSV A Long strain cultured in Hep-2 cells maintained in DMEM medium containing 10% fetal bovine serum was 2.81E+07; the RSV A Long strain is described in the literature: Cultures of HEp-2 cells persistently infected by human respiratory syncytial virus differ in chemokine expression and resistance to apoptosis as compared to lytic infections of the same cell type.

Eight serum samples from each group were diluted in DMEM containing 2% FBS. An equal volume of viral suspension (200 TCID50) was mixed with the diluted serum and incubated at 37° C. for 1 hour. Then, the mixture was added to Hep-2 cell plate at 200 μL per well, with triplicate wells per serum sample. The plates were incubated at 37° C. for 5-7 days, and cytopathic effects (CPE) were observed.

The results were shown in Table 5.

TABLE 5
Neutralization Titers of Different DNA Plasmids

| No. | Encoded Fusion Protein | Mean Neutralization Titer |
|---|---|---|
| 1 | RSV-PreF-A-NP | 713 |
| 2 | RSV-PreF-D-NP | 1017 |
| 3 | RSV-PreF-C-NP | 836 |
| 4 | RSV-PreF-B-NP | 972 |
| 5 | RSV-PreF-E-NP | 643 |
| 6 | RSV-PreF-G-NP | 372 |
| 7 | RSV-PreF-F-NP | 261 |
| 8 | RSV-PreF-H-NP | 130 |

It can be observed that all eight DNA sequences were capable of generating neutralizing titers, with values exceeding 100. DNA-A, DNA-B, DNA-C, and DNA-D (encoding RSV-PreF-A-NP, RSV-PreF-B-NP, RSV-PreF-C-NP, and RSV-PreF-D-NP, respectively) demonstrated superior performance, exhibiting titers greater than 700. This efficacy may be attributed to the specific mutations in the encoded fusion proteins, all of which contain a mutation at position 88.

Based on these results, the four sequences exhibiting the highest neutralization potency were selected for mRNA vaccine preparation.

The mRNA sequences encoding RSV-PreF-A-NP, RSV-PreF-B-NP, RSV-PreF-C-NP, and RSV-PreF-D-NP were obtained by substituting thymine (T) with uracil (U) in SEQ ID No. 20-23.

The coding regions for the Pre-F mutant proteins and ferritin mutants in SEQ ID No. 20-23 were as defined in Table 8.

Similarly, the mRNA sequences encoding RSV-PreF-E-NP, RSV-PreF-F-NP, RSV-PreF-G-NP, and RSV-PreF-H-NP were obtained by replacing thymine (T) with uracil (U) in SEQ ID No. 24-27.

### Example 8. Preparation of mRNA Vaccine

As described in Example 7, plasmid DNA sequences containing RNA transcription related elements were artificially synthesized. The plasmids were transformed into Escherichia coli for amplification. Following fermentation and purification, the plasmids were linearized using restriction endonuclease BspQ1. Uncapped mRNA was obtained through transcription using a T7 in vitro transcription kit. The transcription templates were digested with DNase I, and the mRNA was purified by precipitation. Capping was performed using a Capl capping kit, followed by purification of the capped mRNA with an mRNA purification kit (mRNA sequences encoding RSV-PreF-A-NP, RSV-PreF-B-NP, RSV-PreF-C-NP, and RSV-PreF-D-NP, respectively). The purified mRNA was dissolved in acidic sodium citrate buffer for subsequent use.

Each mRNA sequence was encapsulated into lipid nanoparticles containing cationic lipids using methods known in the prior art to obtain mRNA vaccine (mRNA-LNP), as follows: DLin-MC3-DMA, DSPC, cholesterol, and PEG2000-DMG were dissolved and mixed in ethanol at a molar ratio of 45:10:43:2. The total flow rate of the nanoparticle preparation device was set to 12 mL/min. The mRNA solution and lipid nanoparticle mixture were encapsulated at a flow rate ratio of 3:1. After encapsulation, the samples were collected via tangential flow filtration for buffer exchange, and a sucrose solution was added to obtain the mRNA vaccine (mRNA-LNP). Sampling analysis revealed encapsulation efficiencies >91%, an average particle size of approximately 82 nm, a PDI <0.1, and a Zeta potential of approximately −8.20 mV.

### Example 9. In Vivo Immunogenicity Study

Immunization: On day 0, BALB/c mice were intradermally (i.d.) injected with the mRNA vaccine prepared in Example 8.

Neutralization Titer Assay: The assay was performed using the same method as described in Example 7.

### Neutralization Titer Results

The results were shown in Table 6.

TABLE 6
Neutralization Titer Results of mRNA Vaccines

| No. | Group | Mean Neutralization Titer |
|---|---|---|
| 1 | RSV-PreF-A-NP | 1043 |
| 2 | RSV-PreF-D-NP | 10572 |
| 3 | RSV-PreF-C-NP | 3436 |
| 4 | RSV-PreF-B-NP | 9321 |
| 5 | MRK-12 | 3164 |

It was demonstrated that the mRNA in nanoparticle form of the present invention induced neutralizing protective antibody titers in mice that surpassed those elicited by the concurrently produced MRK-12 (sequence and method derived from SEQ ID No.21 of Moderna's RSV mRNA patent US20230114180A1). Particularly superior results were observed with mRNA vaccines encoding RSV-PreF-D-NP, RSV-PreF-B-NP, and RSV-PreF-C-NP.

### Serum ELISPOT Analysis

The frequency of IFN-γ-secreting cells was measured by intracellular cytokine staining flow cytometry (ICS) following stimulation with the mRNA vaccines (n=5 samples per group).

The results were shown in FIG. 7, after immunization with the RSV-PreF-D-NP mRNA vaccine (RSV-PreF-D-NP group), a mean frequency of 73.4 IFN-γ-secreting cells per 2×10⁵ splenocytes was achieved (5 individual sample values: 58.33, 51, 79, 97.7, and 81). In contrast, the control group MRK-12 showed only 6.4 cells (5 individual sample values: 0, 0.66, 27, 2.33, and 2), demonstrating a statistically significant difference. These ELISPOT cellular immunity results indicated that the mRNA vaccine of the present invention induced significantly stronger cellular immune responses than MRK-12, confirming its superior cellular immunoprotective effects. The cellular immunity elicited may provide more durable and comprehensive protection.

### Example 10. In Vivo Immunogenicity Study of Self-Assembled Nanoparticles

The Catcher and Spy tags were respectively incorporated into ferritin and PreF mutant proteins (refer to Example 6). Following encapsulation methods known in the prior art, both mRNA sequences (mass ratio 1:1) were encapsulated into lipid nanoparticles containing cationic lipid, thereby obtaining mRNA vaccine (mRNA-LNP).

Immunization: On day 0, BALB/c mice were intradermally (i.d.) injected with the mRNA vaccine.

Neutralization Titer Assay: The assay was performed using the same method as described in Example 7.

The neutralization titer results were presented in Table 7.

TABLE 7
Neutralization Titers of Self-assembling mRNA Vaccines

| No. | Group | Mean Neutralization Titer |
|---|---|---|
| 1 | RSV-PreF-A-NP | 1174 |
| 2 | RSV-PreF-D-NP | 10412 |
| 3 | RSV-PreF-C-NP | 2914 |
| 4 | RSV-PreF-B-NP | 8956 |

It was observed that the self-assembled mRNA vaccine exhibited comparable effect to the fusion-expressed construct, with both achieving satisfactory neutralizing titers. Particularly superior results were observed with RSV-PreF-D-NP and RSV-PreF-B-NP, which showed significant differences compared to the other two test groups.

### Example 11. Adjuvant Screening for Ferritin-PreF Fusion Protein

The ferritin-PreF fusion protein (RSV-PreF-D-NP, SEQ ID No. 15) purified in Example 1 (denoted as RSV-NP in the following table) was used. A solvent comprising 20 mM phosphate buffer and 0.15 M NaCl (pH 4.5) was selected to prepare the RSV ferritin-PreF fusion protein antigen.

### I. Grouping

### Vaccine Groups

### 1. Aluminum Adjuvant Preparation

At room temperature, the diluted antigen (0.4 mg/mL) was mixed with aluminum hydroxide adjuvant suspension or aluminum phosphate adjuvant suspension (aluminum content: 10 mg/ml; solute: aluminum hydroxide or aluminum phosphate; solvent: PBS; purchased from Changchun Institute of Biological Products) in glass bottles containing stir bars at 80 rpm/min. Different aluminum salt-adjuvanted vaccines were prepared, with antigen-to-adjuvant mass ratios as shown in Table 9 or Table 10.

### 2. CpG Adjuvant Preparation

At room temperature, the diluted antigen (0.4 mg/mL) was mixed with each 10 mg/mL solution of CpG1018 in Table 11 (solvent: saline; solute: CpG1018; denoted as CpG in Table 11) in glass bottles containing stir bars at 80 rpm/min. Different CpG-adjuvanted vaccines were prepared, with antigen-to-adjuvant mass ratios as shown in Table 11.

### 3. Dual Adjuvant Preparation

At room temperature, the diluted antigen (0.4 mg/mL), aluminum adjuvant (aluminum hydroxide suspension, aluminum content: 10 mg/mL), and CpG solution (10 mg/mL) were mixed in glass bottles containing stir bars at low speed. Different dual-adjuvanted vaccines were prepared, with antigen-to-adjuvant mass ratios as shown in Table 12 or Table 13.

The prepared vaccines were aseptically filled into 2 mL vials (or pre-filled glass syringes), with 0.5 mL (or 1.0 mL) per vial, sealed, and stored protected from light at 2-8° C.

### 4. Adjuvant-Only Group

Prepared using the same method as the dual adjuvant formulation (Group 3), but without antigen.

### 5. Adjuvant-Free Group

Contained antigen only.

### 6. PBS Control Group

PBS preparation: PBS powder (Solarbio, Cat. No.: P1003) was dissolved in 2 L of sterile distilled water per packet.

### II. Immunization

The prepared formulations from section I were used to conduct an immunogenicity study in C57BL/6 mice (purchased from Sibeifu (Beijing) Biotechnology Co., Ltd.).

6-8 weeks-old C57BL/6 mice were randomly divided into groups (n=8 per group). The mice were intramuscularly injected in the thigh with the vaccines in the above each group, setting vaccine groups, adjuvant-free group, and adjuvant-only groups. Prime-boost immunization was performed on day 0 and day 21 (the day of the first immunization was designated as day 0; one week after immunization was recorded as week 1), with a 21-day interval between immunizations. The immunization dose and method were described in section IV.

Blood samples were collected at weeks 3 and 5 post-immunization, and spleens were harvested at week 5.

### III. Detection of Serum Antibodies by ELISA

On day 28 (approximately week 5) post-immunization, mouse serum samples were collected for ELISA analysis. The ELISA procedure was performed as follows: RSV F protein (Sino Biological, Cat. No.: 11049-V08B) was coated at 200 ng per well. Mouse serum samples were used as primary antibodies and were serially diluted 250 fold, 1,250 fold, 6,250 fold, 31,250 fold, 156,250 fold, 781,250 fold, and 3,906,250 fold. A mouse secondary antibody (Cell Signaling Technology, Cat. No.: 7076S) was used as secondary antibody. Absorbance was measured using a microplate reader (Shanghai Kehua, Cat. No.: RD-SH-012).

The antibody titers detected by ELISA in serum samples from mice that received two immunizations were as follows:

### A: Selection of Optimal Aluminum Adjuvant

TABLE 9
ELISA Titers in Mice Immunized with Recombinant Vaccines
Formulated with Different Aluminum Salt Adjuvants

| Group | Immunogen | Adjuvant | Mean ELISA Titer |
|---|---|---|---|
| 1 | PBS | — | <100 |
| 2 | RSV-NP 1 μg | — | 3417 |
| 3 | RSV-NP 1 μg | 50 μg ALPO₄ | 17253 |
| 4 | RSV-NP 1 μg | 50 μg | 51039 (vs. previous |
| | | AL(OH)₃ | group p < 0.01) |

From Table 9, it can be observed that both conventional aluminum adjuvants significantly enhanced antibody titers compared to the adjuvant-free group, with the aluminum hydroxide adjuvant combined with RSV-NP demonstrating superior serum ELISA results.

### B: Screening of Optimal Dosage of Aluminum Adjuvant

TABLE 10
ELISA Titers for Optimal Aluminum Adjuvant Dose Selection

| Group | Immunogen | Adjuvant | Mean ELISA Titer |
|---|---|---|---|
| 5 | PBS | — | <100 |
| 6 | RSV-NP 1 μg | 30 μg | 5423 |
| | | AL(OH)₃ | |
| 7 | RSV-NP 1 μg | 40 μg | 38356 |
| | | AL(OH)₃ | |
| 8 | RSV-NP 1 μg | 50 μg | 51039 (vs. previous group |
| | | AL(OH)₃ | p < 0.01) |
| 9 | RSV-NP 1 μg | 60 μg | 50407 (vs. previous group |
| | | AL(OH)₃ | p > 0.05) |
| 10 | RSV-NP 1 μg | 70 μg | 49179 |
| | | AL(OH)₃ | |

The results were shown in Table 10. Aluminum adjuvant doses ranging from 30-70 μg were all capable of enhancing antibody titers, with the 50 μg dose demonstrating optimal immunogenicity.

### C: Optimal CpG Adjuvant Dosage was Screened

TABLE 11
ELISA Titers for Optimal CpG Adjuvant Dose Selection

| Group | Immunogen | Adjuvant | Mean ELISA Titer |
|---|---|---|---|
| 11 | PBS | — | <100 |
| 12 | RSV-NP 1 μg | 25 μgCpG | 17481 |
| 13 | RSV-NP 1 μg | 50 μgCpG | 22406 (vs. |
| | | | previous group |
| | | | p < 0.01) |
| 14 | RSV-NP 1 μg | 75 μgCpG | 22934 (vs. |
| | | | previous group |
| | | | p > 0.05) |
| 15 | RSV-NP 1 μg | 100 μgCpG | 23047 |

The results were shown in Table 11. Antibody titers were enhanced across the adjuvant dose range of 25-100 μg, with the 50 μg dose CpG1018 adjuvant demonstrating optimal immunogenicity.

### D: Optimal Dual-Adjuvant Dosage was Determined

TABLE 12
ELISA Titers for Optimal Dual-Adjuvant Dose Selection

| Group | Immunogen | Adjuvant | Mean ELISA Titer |
|---|---|---|---|
| 16 | PBS | — | <100 |
| 17 | RSV-NP 1 μg | 50 μg | 148436 |
| | | AL(OH)₃ + 25 μgCpG | |
| 18 | RSV-NP 1 μg | 50 μg | 229309 (vs. |
| | | AL(OH)₃ + 50 μgCpG | previous group |
| | | | p < 0.01) |
| 19 | RSV-NP 1 μg | 50 μg | 230385 (vs. |
| | | AL(OH)₃ + 75 μgCpG | previous group |
| | | | p > 0.05) |
| 20 | RSV-NP 1 μg | 50 μg | 231978 |
| | | AL(OH)₃ + 100 μgCpG | |

The results were presented in Table 12. A plateau in immunogenic response was observed at the 50 μg CpG adjuvant dose, demonstrating statistically significant enhancement of immunological titer compared to the 25 μg dose. Further increasing the dose of CpG adjuvant, the immune titer was maintained at a stable level.

### D: Optimal Antigen Dosage was Investigated

TABLE 13
ELISA Titers for Optimal Antigen Dose in Mice

| Group | Immunogen | Adjuvant | Mean ELISA Titer |
|---|---|---|---|
| 21 | PBS | — | 12359 |
| 22 | RSV-NP | 50 μg | 81498 |
| | 0.25 μg | AL(OH)₃ + 50 μgCpG | |
| 23 | RSV-NP | 50 μg | 125297 |
| | 0.5 μg | AL(OH)₃ + 50 μgCpG | |
| 24 | RSV-NP 1 μg | 50 μg | 229309 (vs. |
| | | AL(OH)₃ + 50 μgCpG | previous group |
| | | | p < 0.01) |
| 25 | RSV-NP 2 μg | 50 μg | 235298 (vs. |
| | | AL(OH)₃ + 50 μgCpG | previous group |
| | | | p > 0.05) |
| 26 | RSV-NP 3 μg | 50 μg | 237898 |
| | | AL(OH)₃ + 50 μgCpG | |

The results were shown in Table 13. It reached a plateau when the antigen dose is approximately 1 μg (P<0.01 for Group 24 vs. Group 23; P>0.05 for Group 24 vs. Groups 25 and 26). Higher antigen doses did not significantly increase ELISA titers.

Conclusion: During vaccine formulation testing, the optimal antigen content was determined to be 1 μg, with 50 μg aluminum hydroxide adjuvant and 50 μg CpG adjuvant identified as the optimal doses.

### IV. CPE Neutralization Assay in Mouse Serum

The TCID50 of RSV A Long strain cultured in Hep-2 cells maintained in DMEM medium containing 10% fetal bovine serum was 2.81E+07; the RSV A Long strain is described in the literature: Cultures of HEp-2 cells persistently infected by human respiratory syncytial virus differ in chemokine expression and resistance to apoptosis as compared to lytic infections of the same cell type. Group 24 from Table 13 was selected, and the corresponding components were removed according to Table 14 to prepare the experimental groups. Eight serum samples per group were diluted in DMEM containing 2% FBS. Serial 3-fold dilutions were performed starting from a 40-fold dilution up to 174,960-fold. The diluted serum was mixed with an equal volume of viral suspension (200 TCID50) and incubated at 37° C. for 1 hour. Then, the mixture was added to Hep-2 cell plate at 200 μL per well, with triplicate wells per serum sample. The plates were incubated at 37° C. for 5-7 days, and cytopathic effects (CPE) were observed.

The results were presented in Table 14. The dual-adjuvant group of the present invention exhibited significantly higher neutralization titers compared to the single-adjuvant groups, with the neutralization titer of 73,152, corresponding Log2 of 16.5 (Table 14).

TABLE 14
Mean Neutralization Titers Against RSV Type A Virus

| No. | Group | Mean Neutralization Titer |
|---|---|---|
| 1 | Dual-Adjuvant Vaccine Group | 73152 |
| 2 | Aluminum Adjuvant Vaccine Group | 5217 |
| 3 | CpG Adjuvant Vaccine Group | 12054 |
| 4 | Adjuvant-Only Control | <64 |
| 5 | Protein-Only Group | 1563 |
| 6 | PBS Control | <100 |

The predominant circulating strains of RSV were classified into types A and B. Following the experimental method described above, the RSV A Long strain was replaced with an RSV B strain (recorded in the literature: Genetic Diversity and Molecular Epidemiology of Circulating Respiratory Syncytial Virus in Central Taiwan, 2008-2017), and the same neutralization titer analysis protocol was performed using the mouse serum samples from each group. The resulting data were presented in Table 15.

TABLE 15
Mean Neutralization Titers Against RSV Type B Virus

| No. | Group | Mean Neutralization Titer |
|---|---|---|
| 1 | Dual-Adjuvant Vaccine Group | 62746 |
| 2 | Aluminum Adjuvant Vaccine Group | 4359 |
| 3 | CpG Adjuvant Vaccine Group | 9183 |
| 4 | Adjuvant-Only Control | <64 |
| 5 | Protein-Only Group | 1031 |
| 6 | PBS Control | <100 |

As shown in Table 15, significantly enhanced neutralization titers were observed in the dual-adjuvant group compared to single-adjuvant groups, demonstrating synergistic effects between the adjuvants.

Similar titer levels and trends were observed with fusion proteins comprising other PreF mutant (eg. RSV-PreF-A-NP (SEQ ID No. 12), RSV-PreF-B-NP (SEQ ID No. 13), RSV-PreF-C-NP (SEQ ID No. 14), RSV-PreF-E-NP (SEQ ID No. 16) etc.)

The present invention has been described above in detail. Those skilled in the art will recognize that, without departing from the spirit and scope of the invention and without undue experimentation, the invention may be practiced over a broad range of equivalent parameters, concentrations, and conditions. Although specific embodiments of the invention have been provided, it should be understood that further modifications and improvements may be made to the invention.

In summary, in accordance with the principles of the present invention, this patent application is intended to encompass any modifications, uses, or improvements of the invention, including changes that extend beyond the disclosed scope and are implemented using conventional techniques known in the art. The application of some essential features may be carried out according to the scope of the appended claims.

1. A biological material, comprising at least one of the following:
(M1) a Pre-F protein mutant, comprising the following mutations on a Pre-F protein amino acid sequence:
serine at position corresponding to 88 of SEQ ID No.1 is substituted with asparagine (S88N);
glutamic acid at position corresponding to 289 of SEQ ID No.1 is substituted with asparagine (E289N);
serine at position corresponding to 309 of SEQ ID No.1 is substituted with asparagine (S309N);
arginine at position corresponding to 468 of SEQ ID No.1 is substituted with asparagine (R468N);
(N1) a fusion protein comprising the Pre-F protein mutant described in (M1) and monomeric subunit protein,
D1) a nucleic acid molecule encoding the Pre-F protein mutant described in (M1) or the fusion protein described in (N1);
D2) an expression cassette comprising the nucleic acid molecule of D1);
D3) a recombinant vector comprising the nucleic acid molecule of D1) or the expression cassette of D2);
D4) a recombinant microorganism comprising the nucleic acid molecule of D1), the expression cassette of D2), or the recombinant vector of D3);
D5) a recombinant cell line comprising the nucleic acid molecule of D1), the expression cassette of D2), or the recombinant vector of D3);
D6) an mRNA, comprising a first open reading frame, wherein the first open reading frame comprises a nucleic acid encoding a monomeric subunit protein and the Pre-F protein mutant described in (M1);
wherein the monomeric subunit protein encoded by the first open reading frame self-assembles into nanoparticles displaying the Pre-F protein mutant on the surface of nanoparticles;
D7) an mRNA, comprising at least two open reading frames, wherein,
a first open reading frame comprises a nucleic acid encoding a monomeric subunit protein; and
a second open reading frame comprises a nucleic acid encoding the Pre-F protein mutant described in (M1);
wherein the protein encoded by the first open reading frame self-assembles into nanoparticles, and the Pre-F protein mutant encoded by the second open reading frame binds to the nanoparticles; D8) a mRNA composition, comprising two nucleic acid molecules, one of which encoding the Pre-F protein mutant described in (M1), the other encoding a monomeric subunit protein;
D9) a protein composition, comprising a Pre-F protein mutant described in (M1) and a monomeric subunit protein.

2. The biological material according to claim 1, wherein, the monomeric subunit protein self-assembles to form nanoparticles, displaying the Pre-F protein mutant on the surface of nanoparticles,
wherein both nucleic acid molecules of mRNA described in D8) are individually labeled with tags, which guide the self-assembly of their encoded peptides into nanoparticles via the interaction of tags, with the Pre-F protein mutant displaying on the surface of nanoparticles;
wherein the monomeric subunit protein and Pre-F protein mutant described in D9) are individually labeled with tags, and the monomeric subunit protein self-assembles to form nanoparticles via the interaction of tags, displaying the Pre-F protein mutant on the surface of nanoparticles,
preferably, wherein the monomeric subunit protein is selected from the group consisting of monomeric ferritin subunit, monomeric encapsulin protein, monomeric 03-33 protein, monomeric sulfoxidase reductase protein, monomeric dihydropteridine synthase protein, and/or monomeric pyruvate dehydrogenase complex dihydrolipoamide acetyltransferase protein.

3. The biological material according to claim 1, wherein,
Pre-F protein amino acid sequence is shown in SEQ ID No. 1 or have at least 95% identity to SEQ ID No. 1 while encoding a protein having the same function;
preferably, the Pre-F protein mutant comprising amino acid sequence of SEQ ID No. 5 or SEQ ID No. 4.

4. The biological material according to claim 1, wherein, the monomeric subunit protein is a ferritin or ferritin mutant,
preferably, wherein the ferritin mutant comprising at least one of the following mutations on an amino acid sequence of a ferritin:
asparagine at position corresponding to 15 of SEQ ID No. 10 is substituted with glutamine;
serine at position corresponding to 96 of SEQ ID No. 10 is substituted with asparagine;
tyrosine at position corresponding to 119 of SEQ ID No. 10 is substituted with arginine.

5. The biological material according to claim 1, wherein, the amino acid sequence of ferritin is shown as SEQ ID No. 10, or have at least 95% identity to SEQ ID No. 10 while encoding a protein having the same function;
preferably, the ferritin mutant comprising an amino acid sequence shown as SEQ ID No.11.

6. The biological material according to claim 1, wherein the fusion protein described in (N1) comprises a linker between the Pre-F protein mutant and the monomeric subunit protein;
preferably, the fusion protein consists of Pre-F protein mutant, linker and ferritin mutant, from N- to C-terminus;
more preferably, the sequence of linker is shown in SEQ ID No.34.

7. The biological material according to claim 1, the fusion protein described in (N1) comprises the amino acid sequence of SEQ ID No. 15 or SEQ ID No. 14.

8. The biological material according to claim 1, wherein, the mRNA encoding fusion protein comprises a nucleic acid sequence shown as SEQ ID No. 33 or 32, or a nucleotide sequence of a degenerate or complementary sequence of SEQ ID No. 33 or 32, or a nucleotide sequence that have at least 95% identity with any one of SEQ ID No. 33 or 32 while retaining the same function;
wherein, the mRNA encoding Pre-F protein mutant comprising a nucleotide sequence of nucleotides 1-1422 or 1-1425 of SEQ ID No.33 or 32, or a degenerate or complementary sequence of nucleotides 1-1422 or 1-1425 of SEQ ID No. 33 or 32, or a nucleotide sequence that have at least 95% identity with nucleotides 1-1422 or 1-1425 of SEQ ID No. 33 or 32 while retaining the same function;
wherein, the mRNA encoding monomeric subunit protein comprising a nucleotide sequence of nucleotides 1477-1941 or 1450-1944 of SEQ ID No.33 or 32; or a nucleotide sequence of a degenerate or complementary sequence of nucleotides 1477-1941 or 1450-1944 of SEQ ID No. 33 or 32, or a nucleotide sequence that have at least 95% identity with nucleotides 1477-1941 or 1450-1944 of SEQ ID No. 33 or 32 while retaining the same function.

9. The biological material according to claim 1, wherein, the nucleic acid molecule encoding the fusion protein described in (N1) comprises a nucleotide sequence of SEQ ID No. 23 or 22, or comprises a nucleotide sequence that have at least 95% identity to SEQ ID No. 23 or 22 while encoding a fusion protein with same function;
wherein, the nucleic acid molecule encoding the Pre-F protein mutant described in (M1) comprises a nucleotide sequence of nucleotides 1-1422 of SEQ ID No. 23 or 22, or comprises a nucleotide sequence that have at least 95% identity to nucleotides 1-1422 of SEQ ID No. 23 or 22 while encoding a protein with the same function.

10. The biological material according to claim 1, wherein the Pre-F protein mutant, the fusion protein, the nucleic acid molecule, the expression cassette, the recombinant vector, the recombinant microorganism, the recombinant cell, or the mRNA or the mRNA composition is used as an immunogen, or used in manufacturing an anti-RSV product, manufacturing a product for preventing and/or treating RSV infection, or manufacturing a product for preventing and/or treating diseases caused by RSV.

11. A pharmaceutical composition, comprising the Pre-F protein mutant, the fusion protein, the protein composition, the nucleic acid molecule, the expression cassette, the recombinant vector, the recombinant microorganism, the recombinant cell, the mRNA, or the mRNA composition of claim 1.

12. The pharmaceutical composition according to claim 11, the Pre-F protein mutant comprising amino acid sequence of SEQ ID No. 5 or SEQ ID No. 4,
the fusion protein described in (N1) comprises the amino acid sequence of SEQ ID No. 15 or SEQ ID No. 14.

13. The pharmaceutical composition according to claim 11, wherein, the mRNA encoding fusion protein comprises a nucleic acid sequence shown as SEQ ID No.33 or 32, or a nucleotide sequence of a degenerate or complementary sequence of any one of SEQ ID No.33 or 32, or a nucleotide sequence that have at least 95% identity with any one of SEQ ID No. 33 or 32, while retaining the same function;
wherein, the mRNA encoding Pre-F protein mutant comprising a nucleotide sequence of nucleotides 1-1422 or 1-1425 of SEQ ID No. 33 or 32, or a degenerate or complementary sequence of nucleotides 1-1422 or 1-1425 of SEQ ID No. 33 or 32, or a nucleotide sequence that have at least 95% identity with nucleotides 1-1422 or 1-1425 of SEQ ID No. 33 or 32 while retaining the same function;
wherein, the mRNA encoding monomeric subunit protein comprising a nucleotide sequence of nucleotides 1477-1941 or 1450-1944 of SEQ ID No. 33 or 32; or a nucleotide sequence of a degenerate or complementary sequence of nucleotides 1477-1941 or 1450-1944 of SEQ ID No. 33 or 32, or a nucleotide sequence that have at least 80% identity with nucleotides 1477-1941 or 1450-1944 of SEQ ID No. 33 or 32 while retaining the same function.

14. The pharmaceutical composition according to claim 11, wherein, the nucleic acid molecule encoding the fusion protein described in (N1) comprises a nucleotide sequence of SEQ ID No. 23 or 22, or comprises a nucleotide sequence that have at least 75% identity to SEQ ID No. 23 or 22 while encoding a fusion protein with same function;
wherein, the nucleic acid molecule encoding the Pre-F protein mutant described in (M1) comprises a nucleotide sequence of nucleotides 1-1422 of SEQ ID No. 23 or 22, or comprises a nucleotide sequence that have at least 75% identity to nucleotides 1-1422 of SEQ ID No. 23 or 22 while encoding a protein with same function.

15. The pharmaceutical composition according to claim 11, further comprising an adjuvant selected from the group consisting of an aluminum adjuvant, CpG adjuvant, or dual adjuvant comprising a combination of an aluminum adjuvant with a CpG adjuvant.

16. The pharmaceutical composition according to claim 15, wherein the aluminum adjuvant comprises one of more of aluminum hydroxide and aluminum phosphate,
preferably, the mass ratio of protein to aluminum adjuvant is 1:(0.5-300), 1:(30-70), 1:(40-70), or 1:(50-60).

17. The pharmaceutical composition according to claim 15, wherein the CpG adjuvant comprises any CpG adjuvant, such as CpG1018, CpG-cjx,
preferably, the mass ratio of protein to CpG adjuvant is 1:(0.5-300), 1:(25-100), or 1:(25-50).

18. The pharmaceutical composition according to claim 15, wherein, in the dual adjuvant, the mass ratio of aluminum adjuvant to CpG adjuvant is (1-5):(5-1); or
the mass ratio of protein to aluminum adjuvant to CpG adjuvant is 1:50:(25-100), or 1:50:(50-100), or 1:50:50, or (0.25-3):50:50, or (1-3):50:50.

19. The pharmaceutical composition according to claim 11, wherein the pharmaceutical composition is used in manufacturing an anti-RSV product, manufacturing a product for preventing and/or treating RSV infection, or manufacturing a product for preventing and/or treating diseases caused by RSV,
preferably, wherein the mRNA is encapsulated in lipid nanoparticles,
more preferably, wherein the lipid nanoparticles comprise cationic lipids, neutral phospholipids, sterol-based lipids, and PEG-lipids,
preferably, the pharmaceutical composition is a vaccine.

20. A method of preventing or treating respiratory syncytial virus infection-related diseases, or a method for inducing an immune response, comprising administering an effective dose of the pharmaceutical composition of claim 11 to a subject in need thereof, thereby inducing an immune response against RSV in the subject.


SEQUENCE LISTING (34 sequences)

SEQ ID NO: 1 - protein, 474 aa, synthetic construct
MELLILKANAITTILTAVTFCFASGQNITEEFYQSTCSAVSKGYLGALRTGWYTSVITIELSNIKEIKCNGTDAKVKLIKQELDKYKSAVTDLQLLMQSTPATGSGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELL

SEQ ID NO: 2 - protein, 474 aa, synthetic construct
MELLILKANAITTILTAVTFCFASGQNITEEFYQSTCSAVSKGYLGALRTGWYTSVITIELSNIKEIKCNGTDAKVKLIKQELDKYKNAVTDLQLLMQSTPATGSGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELL

SEQ ID NO: 3 - protein, 475 aa, synthetic construct
MELLILKANAITTILTAVTFCFASGQNITEEFYQSTCSAVSKGYLGALRTGWYTSVITIELSNIKENKCNGTDAKVKLIKQELDKYKNAVTDLQLLMQSTPATGSGSAIASGVAVCKVLHLEGEVNKIKSALLSTNKAVVSLSGCGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKCRGIIKTFSNGCDYVSNKGVDTVSVGNTLYYVNKQEGQSLYVKGEPIINFYDPLVFPSDEFDASISQVNEKINQSLAFIRKSDELL

SEQ ID NO: 4 - protein, 474 aa, synthetic construct
MELLILKANAITTILTAVTFCFASGQNITEEFYQSTCSAVSKGYLGALRTGWYTSVITIELSNIKEIKCNGTDAKVKLIKQELDKYKNAVTDLQLLMQSTPATGSGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKNGSNICLTRTDRGWYCDNAGNVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFINKSDELL

SEQ ID NO: 5 - protein, 475 aa, synthetic construct
MELLILKANAITTILTAVTFCFASGQNITEEFYQSTCSAVSKGYLGALRTGWYTSVITIELSNIKENKCNGTDAKVKLIKQELDKYKNAVTDLQLLMQSTPATGSGSAIASGVAVCKVLHLEGEVNKIKSALLSTNKAVVSLSGCGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKNGSNICLTRTDRGWYCDNAGNVSFFPQAETCKVQSNRVFCDTMNSRTLPSEVNLCNVDIFNPKYDCKIMTSKTDVSSSVITSLGAIVSCYGKTKCTASNKCRGIIKTFSNGCDYVSNKGVDTVSVGNTLYYVNKQEGQSLYVKGEPIINFYDPLVFPSDEFDASISQVNEKINQSLAFINKSDELL

SEQ ID NO: 6 - protein, 474 aa, synthetic construct
MELLILKANAITTILTAVTFCFASGQNITEEFYQSTCSAVSKGYLGALRTGWYTSVITIELSNIKEIKCNGTDAKVKLIKQELDKYKSAVTDLQLLMQSTPATGSGSAICSGVAVCKVLHLEGEVNKIKSALLSTNKAVVSLSNGVSVLTFKVLDLKNCIDKQLLPILNKQSCSIPNIETVIEFQQKNNRLLEITREFSVNCGVTTPVSTYMLTNSELLSLINDMPITNDQKKLMSNNVQIVRQQSYCIMCIIKEEVLAYVVQLPLYGVIDTPCWKLHTSPLCTTNTKEGSNICLTRTDRGWYCDNAGSVSFFPQAETCKVQSNRVFCDTMNSYTLPSEVNLCEVDIFNPKYDCKIMTSKTDVSSSVITGLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELL

SEQ ID NO: 7 - protein, 475 aa, synthetic construct
MELLILKANAITTILTAVTFCFASGQNITEEFYQSTCSAVSKGYLGALRTGWYTSVITIELSNIKENKCNGTDAKVKLIKQELDKYKSAVTDLQLLMQSTPATGSGSAIASGVAVCKVLHLEGEVNKIKSALLSTNKAVVSLSGCGVSVLTFKVLDLKNYIDKQLLPILNKQSCSIPNIETVIEFQQKNNRLLEITREFSVNCGVTTPVSTYMLTNSELLSLINDMPNTNDQKKLMSNNVQIVRQQSYCIMCIIKEEVLAYVVQLPLYGVIDTPCWKLHTSPLCTTNTKEGSNICLTRTDRGWYCDNAGSVSFFPQAETCKVQSNRVFCDTMNSYTLPSEVNLCEVDIFNPKYDCKIMTSKTDVSSSVITSLGAIVSCYGKTKCTASNKCRGIIKTFSNGCDYVSNKGVDTVSVGNTLYYVNKQEGQSLYVKGEPIINFYDPLVFPSDEFDASISQVNEKINQSLAFIRKSDELL

SEQ ID NO: 8 - protein, 474 aa, synthetic construct
MELLILKANAITTILTAVTFCFASGQNITEEFYQSTCSAVSKGYLGALRTGWYTSVITIELSNIKEIKCNGTDAKVKLIKQELDKYKSAVTDLQLLMQSTPATGSGSAICSGVAVCKVLHLEGEVNKIKSALLSTNKAVVSLSNGVSVLTFKVLDLKNCIDKQLLPILNKQSCSIPNIETVIEFQQKNNRLLEITREFSVNCGVTTPVSTYMLTNSELLSLINDMPITNDQKKLMSNNVQIVRQQSYSIMCIIKEEVLAYVVQLPLYGVIDTPCWKLHTSPLCTTNTKNGSNICLTRTDRGWYCDNAGNVSFFPQAETCKVQSNRVFCDTMNSYTLPSEVNLCEVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFINKSDELL

SEQ ID NO: 9 - protein, 475 aa, synthetic construct
MELLILKANAITTILTAVTFCFASGQNITEEFYQSTCSAVSKGYLGALRTGWYTSVITIELSNIKENKCNGTDAKVKLIKQELDKYKSAVTDLQLLMQSTPATGSGSAIASGVAVCKVLHLEGEVNKIKSALLSTNKAVVSLSGCGVSVLTFKVLDLKCYIDKQLLPILNKQSCSIPNIETVIEFQQKNNRLLEITREFSVNCGVTTPVSTYMLTNSELLSLINDMPITNDQKKLMRNNVQIVRQQSYCIMCIIKEEVLAYVVQLPLYGVIDTPCWKLHTSPLCTTNTKNGSNICLTRTDRGWYCDNAGNVSFFPQAETCKVQSNRVFCDTMNSYTLPSEVNLCNVDIFNPKYDCKIMTSKTDVSSSVITGLGAIVSCYGKTKCTASNKCRGIIKTFSNGCDYVSNKGVDTVSVGNTLYYVNKQEGQSLYVKGEPIINFYDPLVFPSDEFDASISQVNEKINQSLAFINKSDELL

SEQ ID NO: 10 - protein, 165 aa, synthetic construct
DIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 11 - protein, 165 aa, synthetic construct
DIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 12 - protein, 647 aa, synthetic construct
MELLILKANAITTILTAVTFCFASGQNITEEFYQSTCSAVSKGYLGALRTGWYTSVITIELSNIKEIKCNGTDAKVKLIKQELDKYKNAVTDLQLLMQSTPATGSGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELLSGSGGGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 13 - protein, 648 aa, synthetic construct
MELLILKANAITTILTAVTFCFASGQNITEEFYQSTCSAVSKGYLGALRTGWYTSVITIELSNIKENKCNGTDAKVKLIKQELDKYKNAVTDLQLLMQSTPATGSGSAIASGVAVCKVLHLEGEVNKIKSALLSTNKAVVSLSGCGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKCRGIIKTFSNGCDYVSNKGVDTVSVGNTLYYVNKQEGQSLYVKGEPIINFYDPLVFPSDEFDASISQVNEKINQSLAFIRKSDELLSGSGGGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 14 - protein, 647 aa, synthetic construct
MELLILKANAITTILTAVTFCFASGQNITEEFYQSTCSAVSKGYLGALRTGWYTSVITIELSNIKEIKCNGTDAKVKLIKQELDKYKNAVTDLQLLMQSTPATGSGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKNGSNICLTRTDRGWYCDNAGNVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFINKSDELLSGSGGGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 15 - protein, 648 aa, synthetic construct
MELLILKANAITTILTAVTFCFASGQNITEEFYQSTCSAVSKGYLGALRTGWYTSVITIELSNIKENKCNGTDAKVKLIKQELDKYKNAVTDLQLLMQSTPATGSGSAIASGVAVCKVLHLEGEVNKIKSALLSTNKAVVSLSGCGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKNGSNICLTRTDRGWYCDNAGNVSFFPQAETCKVQSNRVFCDTMNSRTLPSEVNLCNVDIFNPKYDCKIMTSKTDVSSSVITSLGAIVSCYGKTKCTASNKCRGIIKTFSNGCDYVSNKGVDTVSVGNTLYYVNKQEGQSLYVKGEPIINFYDPLVFPSDEFDASISQVNEKINQSLAFINKSDELLSGSGGGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 16 - protein, 647 aa, synthetic construct
MELLILKANAITTILTAVTFCFASGQNITEEFYQSTCSAVSKGYLGALRTGWYTSVITIELSNIKEIKCNGTDAKVKLIKQELDKYKSAVTDLQLLMQSTPATGSGSAICSGVAVCKVLHLEGEVNKIKSALLSTNKAVVSLSNGVSVLTFKVLDLKNCIDKQLLPILNKQSCSIPNIETVIEFQQKNNRLLEITREFSVNCGVTTPVSTYMLTNSELLSLINDMPITNDQKKLMSNNVQIVRQQSYCIMCIIKEEVLAYVVQLPLYGVIDTPCWKLHTSPLCTTNTKEGSNICLTRTDRGWYCDNAGSVSFFPQAETCKVQSNRVFCDTMNSYTLPSEVNLCEVDIFNPKYDCKIMTSKTDVSSSVITGLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELLSGSGGGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 17 - protein, 648 aa, synthetic construct
MELLILKANAITTILTAVTFCFASGQNITEEFYQSTCSAVSKGYLGALRTGWYTSVITIELSNIKENKCNGTDAKVKLIKQELDKYKSAVTDLQLLMQSTPATGSGSAIASGVAVCKVLHLEGEVNKIKSALLSTNKAVVSLSGCGVSVLTFKVLDLKNYIDKQLLPILNKQSCSIPNIETVIEFQQKNNRLLEITREFSVNCGVTTPVSTYMLTNSELLSLINDMPNTNDQKKLMSNNVQIVRQQSYCIMCIIKEEVLAYVVQLPLYGVIDTPCWKLHTSPLCTTNTKEGSNICLTRTDRGWYCDNAGSVSFFPQAETCKVQSNRVFCDTMNSYTLPSEVNLCEVDIFNPKYDCKIMTSKTDVSSSVITSLGAIVSCYGKTKCTASNKCRGIIKTFSNGCDYVSNKGVDTVSVGNTLYYVNKQEGQSLYVKGEPIINFYDPLVFPSDEFDASISQVNEKINQSLAFIRKSDELLSGSGGGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 18 - protein, 647 aa, synthetic construct
MELLILKANAITTILTAVTFCFASGQNITEEFYQSTCSAVSKGYLGALRTGWYTSVITIELSNIKEIKCNGTDAKVKLIKQELDKYKSAVTDLQLLMQSTPATGSGSAICSGVAVCKVLHLEGEVNKIKSALLSTNKAVVSLSNGVSVLTFKVLDLKNCIDKQLLPILNKQSCSIPNIETVIEFQQKNNRLLEITREFSVNCGVTTPVSTYMLTNSELLSLINDMPITNDQKKLMSNNVQIVRQQSYSIMCIIKEEVLAYVVQLPLYGVIDTPCWKLHTSPLCTTNTKNGSNICLTRTDRGWYCDNAGNVSFFPQAETCKVQSNRVFCDTMNSYTLPSEVNLCEVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFINKSDELLSGSGGGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 19 - protein, 648 aa, synthetic construct
MELLILKANAITTILTAVTFCFASGQNITEEFYQSTCSAVSKGYLGALRTGWYTSVITIELSNIKENKCNGTDAKVKLIKQELDKYKSAVTDLQLLMQSTPATGSGSAIASGVAVCKVLHLEGEVNKIKSALLSTNKAVVSLSGCGVSVLTFKVLDLKCYIDKQLLPILNKQSCSIPNIETVIEFQQKNNRLLEITREFSVNCGVTTPVSTYMLTNSELLSLINDMPITNDQKKLMRNNVQIVRQQSYCIMCIIKEEVLAYVVQLPLYGVIDTPCWKLHTSPLCTTNTKNGSNICLTRTDRGWYCDNAGNVSFFPQAETCKVQSNRVFCDTMNSYTLPSEVNLCNVDIFNPKYDCKIMTSKTDVSSSVITGLGAIVSCYGKTKCTASNKCRGIIKTFSNGCDYVSNKGVDTVSVGNTLYYVNKQEGQSLYVKGEPIINFYDPLVFPSDEFDASISQVNEKINQSLAFINKSDELLSGSGGGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 20 - DNA, 1941 bp, synthetic construct
  source 1..1941: mol_type = other DNA
atggaactactaatcctaaaggcaaatgcaatcacaaccatcctgaccgccgtgacgttctgcttcgcctctggacagaacatcaccgaggagttctaccagtctacatgctctgctgtgagtaagggctacctgggcgccctgagaaccggctggtacacctctgtcatcaccatcgagctgagcaatatcaaagagatcaagtgcaacggcacagacgccaaagtgaagctgatcaagcaagaactggacaagtacaagaatgctgtgaccgacctgcagctgctgatgcagtccaccccagccaccggctccggctctgccatctgttccggcgtggccgtgtgcaaggtgctgcacctggaaggtgaggtgaataagatcaaaagcgctctgctcagcacaaacaaagctgtggtaagtctcagcaatggcgtgtctgtgctgaccttcaaggtgctggatttgaagaactacatcgacaagcagctgctgcccatcctgaacaagcagtcttgttccatccctaacatcgagacagtgatcgagtttcagcaaaagaacaaccggctgctggaaatcaccagagagttctccgtcaatgctggagtgactacccccgtgtccacctacatgctgaccaactccgagctgctgagcctgattaacgatatgcctatcaccaacgaccagaagaagctgatgtctaacaatgtccagatcgttagacagcagtcctactccatcatgtgcatcatcaaagaagaagtgctggcttatgtggtgcagctgcctctgtacggcgtgatcgacaccccttgctggaagctgcacacctctcctctgtgcaccaccaacaccaaagagggctccaacatttgcctgacacgcacagacagaggctggtactgcgacaacgccggatccgtgagcttcttccctcaggccgaaacctgtaaagttcagtccaacagagtgttctgtgacactatgaactcccggaccctgccatctgaagtgaacctgtgtaacgtggacatcttcaaccccaagtacgactgcaagattatgacctccaaaaccgacgtgtccagctctgtgatcacctccctgggcgctatcgtctcctgctacggcaagaccaagtgtaccgcttctaacaagaaccggggcatcatcaagaccttttccaacggctgcgactacgtgtctaacaagggcgtggataccgtgtccgtgggcaacaccctgtactgcgtgaacaagcaggaaggccagtccctgtacgtgaagggcgagcctatcatcaacttctacgaccccctggtgtttccttccgatgagttcgacgcttctatctctcaagtgaacgagaagatcaaccagtctttggccttcatccggaagtccgacgagctgctgtctggctccggcggcggatctggcgacatcatcaagctgctgaatgaacaggtgaacaaagagatgcaatcttccaatctgtacatgtccatgtcctcctggtgctacacccactctctcgacggcgctggactgtttctgttcgatcacgccgccgaggagtacgagcatgccaaaaagctgattatcttcctgaacgagaacaacgtgcctgtgcagctgacatccatctccgcccctgagcacaagttcgagggcctgacccagatcttccagaaggcctacgaacacgagcagcacatcagcgagagtatcaacaacatcgtggaccacgccatcaagtccaaggaccacgctaccttcaactttctgcagtggtacgtggctgagcaacatgaggaagaggtgctgttcaaggatatcctggataagatcgaactgatcggcaacgagaaccacggcctgtacctagccgatcagtacgtcaagggaatcgccaagtctagaaagtctggctct

SEQ ID NO: 21 - DNA, 1944 bp, synthetic construct
  source 1..1944: mol_type = other DNA
atggaactactaatcctaaaggcaaatgcaataacgaccatcctgaccgctgtgaccttctgcttcgcctcaggccagaacatcacagaggagttctaccagtctacctgttctgccgtttccaagggctacctgggcgccctgcggaccggctggtacacctccgtgatcaccatcgaactgtccaatatcaaagagaacaaatgtaacggcaccgatgccaaagtgaagctgatcaagcaagagctggacaagtacaagaacgccgtgaccgacctgcagctgctgatgcagtctactcctgctacaggctctggctccgccatcgcctctggcgtggccgtgtgcaaggtcctgcacctggaaggtgaggtgaacaagatcaagtctgctctgctgtccaccaacaaggctgtggttagcctgtctggctgcggcgtgtccgtgctgacatttaaggtgctggatctgaagaactacatcgataagcagctgctgcctatcctgaacaagcagtcgtgctctatccctaacatcgagacagtgatcgagtttcagcagaaaaacaaccggctgctggagatcaccagagagttctccgtgaacgccggcgtgactacccccgtcagcacctacatgctgacaaattccgagctgctgtccctaattaacgacatgcctatcaccaacgatcagaagaaactgatgtctaataacgtgcagatcgtgcggcagcaatcctactccatcatgtgcatcatcaaggaagaagtgcttgcttacgtggtgcagctgccactgtatggcgtgatcgacaccccttgttggaagctgcacaccagccctctgtgtaccaccaacaccaaggaaggcagcaacatctgcctgacccgcacagacagaggctggtactgcgacaacgctggctccgtctccttcttccctcaggctgaaacctgcaaggtgcaatccaacagagtgttctgcgataccatgaactctagaaccctgccttccgaggtgaatctgtgcaacgtggacatcttcaaccccaaatacgactgcaagattatgacctccaagaccgacgtgtcctcttctgtgatcacatctctcggcgctatcgtgtcttgctacggcaagaccaagtgcaccgcctccaacaagtgcagaggaatcattaaaaccttctctaatggctgtgactacgtgagcaacaagggagtcgacaccgtgtctgtgggcaataccctgtactacgtcaacaagcaagaaggacaatccctgtatgtgaaaggcgagcccatcatcaacttctacgatcctctggtgttcccctccgacgagttcgacgcctctatctcccaggttaacgagaagatcaaccagtctctggccttcatcagaaagtccgacgagctgctgagtggaagcggcggcggaagcggcgacatcatcaagctgttgaacgagcaggtgaacaaagagatgcagtcctctaacctgtacatgtccatgtcctcctggtgttacacccactccctggacggcgccgggctgttcctgttcgatcacgctgccgaagagtacgagcacgccaagaagctgatcatctttctgaacgaaaataacgtgcctgtgcagctgaccagcatctctgctcctgagcacaagttcgagggcctgacccagatcttccagaaggcttacgagcatgagcagcacatctccgaatctatcaacaacatcgtggaccacgccatcaagtccaaggaccatgctacctttaacttcttgcagtggtacgtggccgagcagcacgaggaagaagtgctgtttaaggacatcctggacaagatcgagctgatcggcaacgagaaccacggcctgtacctggccgatcagtacgtgaagggcatcgccaagagccggaagagtggatct

SEQ ID NO: 22 - DNA, 1941 bp, synthetic construct
  source 1..1941: mol_type = other DNA
atggaactactaatcctaaaggcaaatgcaatcaccacaatcctgacagcagtcacattctgcttcgcctccgggcagaacattaccgaggagttctaccagagcacctgttctgccgtcagcaagggctacctgggagccctgagaaccggatggtacacctccgtgatcaccatcgagctgtccaacatcaaagagatcaagtgcaacggcactgatgctaaggtgaaactgatcaagcaagagctggataagtacaagaacgccgtgaccgacctgcaactgctgatgcagtccacccctgctaccggctccggctctgctatctgctccggcgtggctgtgtgcaaggtgctgcatctggaaggcgaagtgaacaagatcaagtccgctctgctgtccaccaacaaggccgtggtgtctctgtccaatggcgtgtcggtcctgaccttcaaggtgctggatctgaagaactacatcgacaagcaactgctgcctatcctgaacaagcagtcttgctccatcccaaacatcgagaccgtgatcgagttccagcagaagaacaatcggctgctcgaaatcaccagagagttttccgtgaacgccggcgtgaccacacctgtgtccacctacatgctgaccaactctgagctgctgtctctcatcaacgacatgcctatcaccaacgaccagaaaaaactgatgtccaacaacgtgcagatcgtgcggcagcagtcttactccatcatgtgcatcatcaaggaagaagtgctggcctacgtggtgcagctcccactgtatggcgtgatcgacaccccctgctggaaactgcacaccagccctctgtgcacgaccaataccaagaacggctccaacatctgtctgacccggacagacagaggctggtattgtgacaacgctggaaatgtgtctttcttccctcaggccgagacctgtaaagtgcagtccaacagagtgttctgcgacaccatgaactccagaaccctcccctccgaggtgaacctctgtaacgtggatatcttcaaccccaagtacgactgcaagattatgacctccaaaaccgacgtgtccagctccgtcattacatccctgggcgctatcgtgtcctgctacggcaagaccaagtgtaccgccagcaacaagaaccgcggcatcatcaagaccttctccaacggctgcgactacgtgtctaacaaaggcgttgataccgtgtctgtcggcaacaccctgtactgcgtgaacaagcaagagggccagtctctgtacgtgaagggcgagcctatcatcaacttctacgatcctctggtttttccctctgacgagttcgacgcctctatctctcaggtgaatgagaagatcaaccagagcctggccttcatcaataaatctgacgagctgctgtctggctctggcggcggatctggcgacatcatcaagctgctgaacgaacaggtgaacaaagagatgcagtcctctaacctgtacatgtccatgtcttcctggtgctacacacacagcttggacggcgccggattgtttctgttcgaccacgccgctgaagagtacgagcacgccaagaagctgatcattttcctgaacgagaacaacgtgcctgttcagctgaccagcatcagcgctcctgagcacaagttcgagggcctgacccagatctttcagaaggcctacgaacacgagcagcacatctccgaatccatcaacaatatcgtggaccatgccatcaagtccaaggatcacgctacattcaacttcctgcagtggtacgtggccgagcagcacgaggaagaggtgctgttcaaggacattctggacaagatcgaactgatcggcaacgagaaccacggcctgtacctggctgatcagtacgtcaagggcatcgccaagtctcggaagtctggatcg

SEQ ID NO: 23 - DNA, 1944 bp, synthetic construct
  source 1..1944: mol_type = other DNA
atggaactactaatcctaaaggcaaatgcaatcaccacaatcctcaccgctgtgaccttctgcttcgccagcggacagaatatcaccgaggaattttaccagtctacctgttctgctgtctctaaaggctacctgggcgctctgagaaccggctggtacacctccgtgatcaccatcgagctgtctaatatcaaggagaacaagtgtaatggcaccgacgccaaggtgaagctgatcaagcaagagctggacaagtacaagaacgccgtgaccgatctgcagctgctgatgcagtccacccctgctactggctctggctccgccatcgcctctggcgtggccgtgtgcaaggtgctgcacctggaaggcgaggtcaacaagatcaagtccgccctgctgtctaccaacaaggctgtggtctccctgtccggctgtggcgtgagcgtgctgacattcaaggtcctggacctcaagaactacatcgacaagcagctcctgcctatcctcaacaagcagtcctgctccatccctaacatcgaaaccgtgatcgagtttcagcagaaaaacaaccggctgctggaaatcacaagagagttctccgtcaatgccggcgtgacaaccccagtgagcacctacatgctgacaaactccgaactgctgagcttgatcaacgacatgcccatcaccaacgaccagaaaaagctgatgtccaacaatgtgcagatcgtgcggcagcagtcctactccatcatgtgcatcatcaaagaagaggttctagcttacgtggtgcaactgcccctgtatggagtgattgacaccccttgctggaagctgcatacatctcctctgtgtactaccaataccaagaacggttccaacatctgcctgaccagaaccgaccggggctggtactgcgacaacgctggcaacgtgtccttctttcctcaggccgagacatgtaaggtgcagtccaacagagtgttctgtgacaccatgaactctcggaccctgccttctgaggtgaacctgtgcaacgtggacatcttcaaccccaagtacgactgcaagattatgacctctaagacagacgtgtcttcgtccgtgatcacctctctgggcgccatcgtgtcctgctacggcaagaccaagtgcaccgcctccaacaagtgcagaggcatcatcaagacgtttagcaacggctgcgattacgtcagcaacaagggcgtggataccgtgtccgtgggcaacaccctgtactacgtgaataaacaagagggccagtctctgtacgtgaagggagagcctatcatcaacttctacgaccctctggtgttcccctccgacgagttcgatgcctctatctcgcaagttaacgagaagatcaaccagtccctggccttcatcaacaagtctgacgagctgctgtccggttccggcggaggctctggcgacatcatcaaactgctgaacgaacaggtgaacaaagaaatgcagagctctaacctgtacatgtccatgtcttcttggtgctacacccactccttggatggcgctggactgtttctgttcgaccacgccgctgaggagtacgagcacgctaagaagctgatcatcttcctgaacgaaaacaacgtgcctgtgcagctgaccagcatcagcgctcctgaacacaagttcgagggcctgacccagatcttccagaaggcctacgagcacgagcagcacatctccgagtccatcaacaacattgtggatcacgccatcaagtccaaggaccatgccaccttcaacttcctgcagtggtatgtggctgagcagcacgaggaagaagtgctgttcaaggatatcctggataagatcgagctgatcggcaacgagaaccacggcctgtacctggccgaccagtacgtgaaaggcatcgccaagtctagaaaatccggctct

SEQ ID NO: 24 - DNA, 1941 bp, synthetic construct
  source 1..1941: mol_type = other DNA
atggagctgttgatcctgaaggcaaatgctatcacaaccatcctgaccgccgttactttctgttttgcttccggccagaacatcactgaggagttttatcagagcacctgctccgcagtcagtaagggatatttgggcgccctcagaacagggtggtatacatctgttatcacaatcgaactttctaacattaaagaaatcaaatgtaatggcactgacgccaaggtgaagctcatcaagcaggaactcgacaagtataagagtgctgttactgatctgcaactgctgatgcaaagtaccccagccacaggcagtggaagtgctatatgtagcggcgtcgctgtgtgcaaagtgcttcacctcgagggggaggttaacaaaatcaaatccgccctcctgagcacaaataaggctgtggtgagtttgagcaacggcgtgagcgtgctgacctttaaggttctggacttgaaaaactgtattgacaaacagctgctgcctattttgaacaagcagtcttgctcaatccccaacatcgagacagtaatagaatttcaacaaaagaataaccggctcctggagatcacacgtgaattcagcgtgaactgcggtgttacaacccccgtttctacctatatgctcacaaacagtgagctgctttctctgatcaacgacatgcctatcaccaacgatcagaagaaacttatgagcaataacgtgcagatcgttcgccagcagagctactgtatcatgtgcatcataaaagaggaggtcctcgcctatgtggtgcagctcccattgtacggcgtgatagataccccctgttggaaactgcatacatcccctttgtgtaccacaaacacaaaagagggcagcaacatctgcctcactaggactgacagaggttggtattgcgacaatgccgggtcagtctcatttttcccacaagctgagacctgtaaagtccagtccaaccgcgtgttctgcgatacaatgaactcatataccctgccaagcgaagtgaacctctgcgaggtcgatattttcaaccctaagtacgattgtaaaattatgacttctaagactgacgtaagttctagcgtcataaccggactgggcgccatagtgtcctgctacggcaaaaccaagtgcactgcaagcaataagaatagagggatcattaaaaccttctccaacggctgtgattacgtgtctaataaaggagtggacacagtgagtgtgggtaacactctgtattgtgtgaacaagcaggagggacagtcactctacgtcaagggagaaccaatcatcaatttttacgaccccctcgtgttcccctcagatgaatttgacgccagcatttcccaggtgaacgagaaaataaatcagtctctggcctttattagaaaatccgatgaactgctgagcggcagtggaggcggttctggagacatcatcaagctgctcaatgagcaagtgaacaaggagatgcagtcttctaacctgtacatgagcatgtcttcctggtgttacacccactctctcgatggtgccggcctgttcctgtttgaccatgctgccgaggagtacgagcacgctaaaaagctgatcatatttctgaacgaaaataacgttcccgtacagcttaccagtattagcgcccccgagcataagttcgaaggtttgacccagatctttcagaaggcctatgagcacgagcagcatatttccgagtccatcaataatattgtagaccacgccatcaagtctaaagaccatgctacttttaacttcctgcaatggtacgtggctgagcagcacgaggaggaggtactctttaaggacattctcgacaagattgagcttatcggaaacgagaatcatggtctctacctggctgatcagtatgtcaagggcattgccaagagtaggaagagtggcagc

SEQ ID NO: 25 - DNA, 1944 bp, synthetic construct
  source 1..1944: mol_type = other DNA
atggagctgctgatactgaaagcaaacgccatcacaacaatactcaccgctgtcactttctgtttcgccagcggacagaatattacagaggagttctaccagagcacttgtagtgctgtctctaaggggtatctgggcgcactgaggaccggatggtatacaagcgtgatcactattgagctgagtaacattaaggagaataagtgcaatggcacagacgccaaagtcaagttgatcaagcaggagctcgacaagtataaatccgccgtgaccgacctgcagctcctcatgcagtctacacctgctacagggtctggatcagcaatagcatctggagtggcagtatgtaaggtgctgcatctggaaggagaggtaaataaaatcaagtcagcccttctgagcactaataaggctgtggtgagcctgagtggatgcggtgtcagcgtcctgactttcaaggtgttggacttgaagaactacatcgacaagcaactgctgcctattttgaacaaacagagttgtagcatccctaatatcgagactgtgatcgagtttcaacagaagaacaacaggctgcttgagattacacgtgaattttctgtgaactgcggcgtgaccacccctgtatccacatacatgctgacaaactccgagcttctttctctgattaatgacatgcctaacacaaatgaccagaagaagctgatgtccaacaatgtgcaaattgtacggcagcagtcctattgtatcatgtgtatcatcaaggaagaggttctcgcatacgtggtacaactcccactgtacggcgttatcgatactccatgttggaagctgcatactagccctctttgtactaccaacaccaaggaggggagcaacatctgtctcacccgcaccgatcggggttggtattgcgataacgccggaagcgtcagcttctttcctcaggcagagacttgcaaagtgcagtctaacagagttttttgcgacacaatgaattcctacaccttgccaagcgaagtaaacctctgcgaagtggatattttcaaccctaaatacgactgcaagatcatgacctccaagaccgatgtgagcagcagcgttatcactagtctcggagctattgtatcctgctatggaaagacaaagtgcaccgccagcaataaatgtcggggaatcatcaagactttttccaacggttgcgactatgtgagtaataagggtgtggatacagtatcagttggcaacacactgtactacgtgaacaaacaggaaggccagtctctctacgtgaagggtgaacccataattaatttttatgaccctttggtgtttcctagtgatgagtttgacgcctctatcagccaggtgaatgaaaagattaatcagtccctggcctttatacgcaagagcgatgagcttctgagcggaagtggcggaggcagtggagatattattaagttgctgaatgaacaagtaaataaggaaatgcaatcttctaatttgtatatgtctatgagcagttggtgctatactcactcactggacggggctggcctctttctctttgatcatgctgctgaggaatacgagcatgccaagaagctgataatcttcttgaacgaaaacaacgtgcccgtgcagcttaccagcattagcgcccccgagcacaagttcgaaggcctcacccagatctttcagaaggcatacgagcacgagcagcacatttcagagagtattaataacatcgtcgaccacgccattaaaagtaaggatcatgctacctttaattttctgcaatggtacgtggccgagcagcatgaagaggaagtgctgttcaaggacatcctggacaagatcgaactgatcggcaacgagaaccacggtctctacttggccgaccaatatgtgaagggaatagccaaaagtagaaagagcggctct

SEQ ID NO: 26 - DNA, 1941 bp, synthetic construct
  source 1..1941: mol_type = other DNA
atggagctgctgattctgaaagccaatgccattaccacaatcctgacagctgttacattctgcttcgcttccgggcagaatatcaccgaggagttctatcagagtacttgttctgccgtgtcaaaagggtatttgggcgccctgcgaacaggatggtacacatcagttatcaccattgagcttagcaatatcaaggagattaagtgcaatggaaccgacgccaaggtgaagctgattaagcaggaacttgacaagtacaaatcagcagtgacagatttgcagctgttgatgcaatccacacccgctaccggatcagggagtgccatttgttctggcgtagctgtgtgcaaagtgctccatctggaaggagaagtgaataaaattaaaagcgccctcctgagcacaaataaggcagtggtgtccctcagtaacggagtgtctgtgctgacattcaaggtgctggacctgaagaattgtatagataagcaactgctgcccatccttaataaacagtcatgcagcattcctaatatcgagaccgtgatcgagtttcaacagaaaaacaaccgccttttggagattactcgggagttctccgtgaattgcggtgtcaccactcccgtgtccacctatatgcttacaaactcagagctcctttcactgatcaatgacatgccaattacaaatgatcagaaaaagctgatgtctaacaatgtccagatcgtgcgacagcagagctactcaattatgtgtatcattaaagaggaggtcttggcttatgtcgtgcagttgcctctgtatggcgtcattgacactccctgttggaaactgcacacttcacccctctgtaccactaacaccaagaacgggtctaacatctgtttgacaaggaccgatcggggctggtattgtgacaacgctggaaacgtcagctttttccctcaagccgaaacatgtaaggtccagtcaaatagagtgttttgtgacacaatgaactcttatacactgccaagcgaggtgaatctctgcgaagttgatattttcaaccctaagtacgactgtaagattatgacctcaaaaactgatgtgtcttcaagcgtgattacaagcctgggcgccatcgtgtcttgctacggcaagaccaagtgtacagcctctaacaaaaacaggggaatcattaaaaccttttcaaatggttgcgactatgtgtccaataagggcgttgatactgtcagcgtaggcaacacattgtattgtgtcaacaagcaggagggtcagagcctgtacgtgaagggtgaacctatcattaacttctacgacccccttgtcttccctagtgatgagtttgatgcttcaatctcacaggtaaatgagaagatcaaccagtccttggctttcattaacaagagtgatgaactgctgtccggttctggaggcggctcaggtgatattatcaaactccttaatgagcaagtcaacaaagagatgcaaagctccaatctgtatatgtccatgtcttcctggtgctacacccattccctggacggagcaggcctgttcttgtttgaccacgcagccgaggagtatgagcacgccaagaagctgattatcttcttgaacgagaacaacgtgccagtccagctgactagtatttctgccccagaacacaagttcgaaggcttgactcaaatcttccagaaagcatacgagcacgagcaacatatcagcgagagtattaacaatatcgtcgatcacgccattaagagtaaagaccacgcaactttcaacttcctccagtggtatgtggccgagcagcacgaggaggaagtgctgttcaaagatattttggataaaatcgagcttattggaaacgagaaccatggtctctatctggcagatcagtacgtgaaggggattgcaaaatctagaaaatctggctcc

SEQ ID NO: 27 - DNA, 1944 bp, synthetic construct
  source 1..1944: mol_type = other DNA
atggagttgctcatactgaaggctaacgctatcaccaccatcttgaccgccgtcaccttctgtttcgcttccggacagaacatcaccgaggagttctaccagagtacatgttctgccgtgtctaagggctatctgggcgccctccgcactggctggtacacaagtgtcattactatcgagctgtccaatatcaaggaaaataagtgcaacggcaccgatgcaaaagtgaaacttatcaaacaggaactggataagtataagagcgctgtgactgatctgcagctgctcatgcagtcaacacctgccactggtagcggtagtgctatcgcatctggtgttgccgtgtgtaaagttctgcatctggagggcgaggtgaataagatcaagtctgcactcctctccactaacaaagccgtcgtcagcctttccggctgtggcgtgtctgttctcacttttaaggtcctcgacctcaaatgttacattgataagcagctgctgcccattctcaacaaacagtcctgctctatccctaatatcgaaactgtcattgagtttcagcaaaaaaacaaccgacttctcgagattactcgtgaattctcagtcaattgtggtgtcaccaccccagtttccacctatatgctgacaaattctgagctgctcagcctcatcaatgacatgccaatcaccaacgatcagaagaaattgatgagaaataacgtgcagatcgtacgacaacagagttactgtattatgtgtatcatcaaagaggaagtgcttgcctacgtggtacagctccctctttacggggtaattgacacaccatgctggaagctgcacacatctccattgtgtaccactaacaccaagaatggctccaacatctgcttgacacggaccgacagaggctggtactgcgacaacgccgggaacgtgtcttttttccctcaggctgaaacatgcaaggtgcaaagcaatagagtcttctgcgatactatgaatagctataccctgcccagcgaggtcaatctgtgcaacgtggacatttttaatccaaagtatgattgcaagatcatgaccagcaagaccgacgtatcttcaagcgttatcactggacttggcgccatagttagctgctatggcaagaccaaatgtacagccagtaacaagtgccggggtatcattaaaacctttagcaacggctgcgactatgtgagcaataagggcgtagacacagtatctgtgggaaatacattgtactacgtcaacaaacaggagggtcagtccctctacgtcaagggtgagcccattatcaatttttatgaccccctggtcttcccttcagatgagttcgacgccagcattagtcaggtgaatgagaaaattaatcagtcactggcattcatcaacaagagtgacgaactgctgtccggcagcggagggggaagcggggacatcattaagctgctgaacgaacaagtgaacaaggagatgcagtcctccaacctgtacatgtcaatgtcttcttggtgttacacacactctctggatggggccggtctttttctttttgaccacgccgcagaggaatacgaacacgctaagaagctgattatcttccttaacgagaacaacgtgcctgtgcagctgacctccatctccgcacctgagcataagttcgagggcctcactcagatttttcagaaggcatacgaacatgagcagcacatttccgagtcaatcaacaatattgtcgatcacgcaatcaagtccaaggatcacgcaactttcaactttttgcagtggtacgtggccgagcagcatgaagaggaagtcctgtttaaggacatcctcgacaagatcgaactgatcggcaacgagaatcatggcctttacttggccgatcagtacgtgaaaggaatagctaagagcagaaagagcggatct

SEQ ID NO: 28 - protein, 116 aa, synthetic construct
GAMVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 29 - protein, 16 aa, synthetic construct
RGVPHIVMVDAYKRYK

SEQ ID NO: 30 - RNA, 1941 nt, synthetic construct
  source 1..1941: mol_type = other RNA
atggaactactaatcctaaaggcaaatgcaatcacaaccatcctgaccgccgtgacgttctgcttcgcctctggacagaacatcaccgaggagttctaccagtctacatgctctgctgtgagtaagggctacctgggcgccctgagaaccggctggtacacctctgtcatcaccatcgagctgagcaatatcaaagagatcaagtgcaacggcacagacgccaaagtgaagctgatcaagcaagaactggacaagtacaagaatgctgtgaccgacctgcagctgctgatgcagtccaccccagccaccggctccggctctgccatctgttccggcgtggccgtgtgcaaggtgctgcacctggaaggtgaggtgaataagatcaaaagcgctctgctcagcacaaacaaagctgtggtaagtctcagcaatggcgtgtctgtgctgaccttcaaggtgctggatttgaagaactacatcgacaagcagctgctgcccatcctgaacaagcagtcttgttccatccctaacatcgagacagtgatcgagtttcagcaaaagaacaaccggctgctggaaatcaccagagagttctccgtcaatgctggagtgactacccccgtgtccacctacatgctgaccaactccgagctgctgagcctgattaacgatatgcctatcaccaacgaccagaagaagctgatgtctaacaatgtccagatcgttagacagcagtcctactccatcatgtgcatcatcaaagaagaagtgctggcttatgtggtgcagctgcctctgtacggcgtgatcgacaccccttgctggaagctgcacacctctcctctgtgcaccaccaacaccaaagagggctccaacatttgcctgacacgcacagacagaggctggtactgcgacaacgccggatccgtgagcttcttccctcaggccgaaacctgtaaagttcagtccaacagagtgttctgtgacactatgaactcccggaccctgccatctgaagtgaacctgtgtaacgtggacatcttcaaccccaagtacgactgcaagattatgacctccaaaaccgacgtgtccagctctgtgatcacctccctgggcgctatcgtctcctgctacggcaagaccaagtgtaccgcttctaacaagaaccggggcatcatcaagaccttttccaacggctgcgactacgtgtctaacaagggcgtggataccgtgtccgtgggcaacaccctgtactgcgtgaacaagcaggaaggccagtccctgtacgtgaagggcgagcctatcatcaacttctacgaccccctggtgtttccttccgatgagttcgacgcttctatctctcaagtgaacgagaagatcaaccagtctttggccttcatccggaagtccgacgagctgctgtctggctccggcggcggatctggcgacatcatcaagctgctgaatgaacaggtgaacaaagagatgcaatcttccaatctgtacatgtccatgtcctcctggtgctacacccactctctcgacggcgctggactgtttctgttcgatcacgccgccgaggagtacgagcatgccaaaaagctgattatcttcctgaacgagaacaacgtgcctgtgcagctgacatccatctccgcccctgagcacaagttcgagggcctgacccagatcttccagaaggcctacgaacacgagcagcacatcagcgagagtatcaacaacatcgtggaccacgccatcaagtccaaggaccacgctaccttcaactttctgcagtggtacgtggctgagcaacatgaggaagaggtgctgttcaaggatatcctggataagatcgaactgatcggcaacgagaaccacggcctgtacctagccgatcagtacgtcaagggaatcgccaagtctagaaagtctggctct

SEQ ID NO: 31 - RNA, 1944 nt, synthetic construct
  source 1..1944: mol_type = other RNA
atggaactgctgatcctcaaggccaacgccattaccaccatcctgaccgccgtgaccttctgcttcgccagcggacagaacatcaccgaggaattttaccagtctacatgttctgccgtgtccaagggctacctgggcgctctgcggaccggctggtacaccagcgtgattaccatcgagctgagcaatatcaaggaaaacaagtgcaacggcaccgacgccaaggtgaagctgattaagcaggagctggataagtacaagaacgccgtgaccgatttgcagctgctgatgcagagcacccctgccacaggatctggaagcgctatcgcctctggcgtggctgtgtgcaaggtgctgcacctggaaggggaggtgaacaagatcaaaagcgccctgctgtctacaaacaaagccgtcgtgtccctcagcggctgtggcgtctccgtgctgactttcaaggtgctggacctgaagaattacatcgacaagcaactgctgcccatcctgaacaagcagagctgcagcatacccaacatcgagacagtgattgaattccaacaaaaaaacaaccggctgctggaaatcaccagagagttcagcgtgaatgctggcgtgacaacaccagtgagcacatacatgctgacaaatagcgaactgctgagcctgatcaacgacatgcctatcaccaacgaccagaagaaactgatgagcaacaatgtgcagatcgtgcggcagcagtcctacagcatcatgtgcatcatcaaggaagaggtgcttgcttacgtggtgcagctgcctctgtatggcgtgatcgacaccccctgctggaagctccatacaagccctctgtgtaccacaaacaccaaggaaggtagcaatatctgcctgacaagaaccgatagaggctggtattgtgacaacgccggctctgtgtccttcttcccacaggccgaaacctgtaaagtgcagagcaatagagtgttctgcgacacaatgaacagccggaccctgccttctgaggttaacctgtgcaatgtggatatctttaaccccaagtacgactgcaagatcatgaccagcaagacagatgtgtcatcgagcgtcatcaccagcctgggagctatcgtgtcctgctacggcaagaccaagtgcaccgcctctaataaatgtagaggcatcatcaagacctttagcaacggctgcgactacgtgagcaacaagggcgttgataccgtgagcgtgggcaacaccctgtactacgtgaacaagcaggagggccagagcttatatgtgaagggcgaacctatcatcaatttctacgatcctctggtgttccccagcgacgagttcgacgcctctatttctcaagtgaacgagaagatcaaccagagcctggccttcatcagaaagtccgatgagctgctgtctggctccggcggaggcagcggcgacatcatcaaactgctgaacgaacaggtgaacaaagagatgcagagcagcaatctgtacatgagcatgtcttcctggtgctacacacacagcctggacggcgccggcctgttcctgtttgaccacgccgccgaggagtacgagcatgctaaaaagctgatcatctttctgaacgagaacaacgtgcctgtgcaactgaccagcatcagcgcccctgagcacaagttcgagggcctgacgcagatcttccagaaggcctacgagcacgagcagcacatcagcgaaagcatcaacaacatcgtcgatcacgctatcaagtctaaagaccacgccaccttcaacttcctgcagtggtacgtggccgagcagcacgaggaagaagtgctgttcaaggacatcctggacaagatcgagctgatcggcaacgagaaccacggcctgtacctggccgaccagtacgtcaagggaatcgccaaaagcagaaagagcggctcc

SEQ ID NO: 32 - RNA, 1941 nt, synthetic construct
  source 1..1941: mol_type = other RNA
atggaactactaatcctaaaggcaaatgcaatcaccacaatcctgacagcagtcacattctgcttcgcctccgggcagaacattaccgaggagttctaccagagcacctgttctgccgtcagcaagggctacctgggagccctgagaaccggatggtacacctccgtgatcaccatcgagctgtccaacatcaaagagatcaagtgcaacggcactgatgctaaggtgaaactgatcaagcaagagctggataagtacaagaacgccgtgaccgacctgcaactgctgatgcagtccacccctgctaccggctccggctctgctatctgctccggcgtggctgtgtgcaaggtgctgcatctggaaggcgaagtgaacaagatcaagtccgctctgctgtccaccaacaaggccgtggtgtctctgtccaatggcgtgtcggtcctgaccttcaaggtgctggatctgaagaactacatcgacaagcaactgctgcctatcctgaacaagcagtcttgctccatcccaaacatcgagaccgtgatcgagttccagcagaagaacaatcggctgctcgaaatcaccagagagttttccgtgaacgccggcgtgaccacacctgtgtccacctacatgctgaccaactctgagctgctgtctctcatcaacgacatgcctatcaccaacgaccagaaaaaactgatgtccaacaacgtgcagatcgtgcggcagcagtcttactccatcatgtgcatcatcaaggaagaagtgctggcctacgtggtgcagctcccactgtatggcgtgatcgacaccccctgctggaaactgcacaccagccctctgtgcacgaccaataccaagaacggctccaacatctgtctgacccggacagacagaggctggtattgtgacaacgctggaaatgtgtctttcttccctcaggccgagacctgtaaagtgcagtccaacagagtgttctgcgacaccatgaactccagaaccctcccctccgaggtgaacctctgtaacgtggatatcttcaaccccaagtacgactgcaagattatgacctccaaaaccgacgtgtccagctccgtcattacatccctgggcgctatcgtgtcctgctacggcaagaccaagtgtaccgccagcaacaagaaccgcggcatcatcaagaccttctccaacggctgcgactacgtgtctaacaaaggcgttgataccgtgtctgtcggcaacaccctgtactgcgtgaacaagcaagagggccagtctctgtacgtgaagggcgagcctatcatcaacttctacgatcctctggtttttccctctgacgagttcgacgcctctatctctcaggtgaatgagaagatcaaccagagcctggccttcatcaataaatctgacgagctgctgtctggctctggcggcggatctggcgacatcatcaagctgctgaacgaacaggtgaacaaagagatgcagtcctctaacctgtacatgtccatgtcttcctggtgctacacacacagcttggacggcgccggattgtttctgttcgaccacgccgctgaagagtacgagcacgccaagaagctgatcattttcctgaacgagaacaacgtgcctgttcagctgaccagcatcagcgctcctgagcacaagttcgagggcctgacccagatctttcagaaggcctacgaacacgagcagcacatctccgaatccatcaacaatatcgtggaccatgccatcaagtccaaggatcacgctacattcaacttcctgcagtggtacgtggccgagcagcacgaggaagaggtgctgttcaaggacattctggacaagatcgaactgatcggcaacgagaaccacggcctgtacctggctgatcagtacgtcaagggcatcgccaagtctcggaagtctggatcg

SEQ ID NO: 33 - RNA, 1944 nt, synthetic construct
  source 1..1944: mol_type = other RNA
atggaactgctgatcctgaaggccaacgccattaccacaatcctgaccgccgtgaccttttgttttgcctctggccagaacattaccgaagaattctaccagtccacatgttctgccgtgtccaagggctacctcggcgctcttagaacaggctggtatacaagcgtgatcacaatcgaactcagcaatatcaaagaaaacaagtgcaacggtacagacgccaaggtgaaactgatcaagcaggagctggacaagtacaagaacgccgtgaccgacctgcaactgctgatgcagagcacccctgctaccggctccggcagtgcaatcgccagcggggtggccgtgtgcaaagtgctgcacctggaaggcgaggtgaacaaaatcaagagcgccctgctgagcacaaacaaagccgttgtgtcactgtctggctgcggcgtcagcgtgctgacctttaaggtgctggacctgaaaaactacatcgacaagcaactgctgcctatcctgaacaagcaatcttgcagcatcccgaacatcgagacagtgattgagttccaacagaagaataacagactgctggaaatcactcgggaattcagcgtgaatgccggagttaccacccctgtgagcacctacatgctgaccaactctgagctgctgagcctgataaacgacatgcctatcaccaatgatcagaagaagctgatgagcaacaacgtgcagatcgtgcggcagcagtcctacagcatcatgtgcatcatcaaggaagaagtgctggcttacgtggtgcaactgcctctgtatggcgtgatcgatacaccttgttggaaactgcacacctctcctctgtgcacaaccaacaccaagaacggcagcaacatctgcctgaccagaaccgacagaggctggtactgcgacaacgccggcaacgtgagcttcttcccccaggccgagacatgcaaggtccagtccaatcgggtgttctgcgacaccatgaacagcaggaccctgcccagcgaggtgaacctgtgtaacgtcgatatcttcaaccccaagtacgattgcaaaatcatgaccagcaagaccgacgtgtccagcagcgtgatcaccagcctgggcgctatcgttagctgctacggcaagaccaagtgcaccgccagcaacaagtgtagaggcatcatcaaaacattcagcaatggctgtgattacgtgtccaacaagggcgtggacaccgtgtctgttggaaacacactgtactacgtgaataagcaggagggccagagcctgtacgtgaagggagaacctatcatcaacttctacgatcccctcgtgttcccaagcgacgaattcgacgccagcatctctcaggtgaacgagaagatcaatcagtctctggccttcatcaacaagtctgatgagctgctgagcggctccggaggcggaagcggcgacatcatcaagctgctcaacgaacaggtgaacaaagagatgcagtcctctaatctgtacatgagcatgtctagctggtgctacacacacagcctggacggcgccggcctgttcctgtttgatcatgctgctgaagagtacgagcacgctaaaaagctgatcatcttcctgaatgaaaacaacgtcccagtgcagctgacaagcatcagcgcccctgagcacaagttcgagggcctgacccagatctttcagaaagcctatgagcacgagcagcacatcagcgagagcatcaacaacatcgtggaccacgccatcaagagcaaggaccacgccacctttaacttcctgcagtggtacgtggccgagcagcatgaggaggaagtgctgttcaaggacatactggacaagatcgagctgatcggaaatgagaatcacggcctctatctggccgatcagtacgtgaagggaattgccaagtccagaaagagcggctct

SEQ ID NO: 34 - protein, 8 aa, synthetic construct
SGSGGGSG